(19) United States
(12) Patent Application Publication
(10) Pub. No.: US 2008/0064957 A1
(43) Pub. Date: Mar. 13, 2008

(54) ULTRASONIC IMPLANT, SYSTEMS AND METHODS RELATED TO DIVERTING MATERIAL IN BLOOD FLOW AWAY FROM THE HEAD

(76) Inventor: Paul A. Spence, Louisville, KY (US)

Correspondence Address: WOOD, HERRON & EVANS, LLP, 2700 CAREW TOWER, 441 VINE STREET, CINCINNATI, OH, 45202, US

(21) Appl. No.: 11/851,793

(22) Filed: Sep. 7, 2007

Related U.S. Application Data
(60) Provisional application No. 60/824,842, filed on Sep. 7, 2006.
(60) Provisional application No. 60/826,960, filed on Sep. 26, 2006.

(51) Int. Cl. A61M 29/00; A61B 18/02; A61N 7/00

(52) U.S. Cl. 600/439; 606/200; 606/21

(57) ABSTRACT

A system for directing ultrasonic energy through blood flow in the aorta of a patient proximate the origins of the great vessels to the head of the patient to divert material in the blood flow from the origins of the great vessels. The system comprises a first device configured for introduction into the cardiovascular system of the patient and positioning proximate the origins of the great vessels to the head of the patient, and at least one ultrasonic energy emitter carried by said first device.

This application claims the benefit of U.S. Provisional Patent Application Ser. Nos. 60/824,842 and 60/826,960, respectively filed on Sep. 7, 2006 and Sep. 26, 2006. The full disclosures of U.S. Provisional Patent Application Ser. Nos. 60/824,842 and 60/826,960 are expressly incorporated by reference herein.

### TECHNICAL FIELD

The present invention generally relates to apparatus and methods involving the use of ultrasound to divert material, such as particulates or air bubbles in the blood flow away from arteries leading to the head of a patient.

### BACKGROUND

Stroke is a major cause of death and disability worldwide. Often, stroke is feared more than other types of disease, or even death, because it leaves the patient in a permanent state of dependency requiring constant care by family or other caregivers. Stroke occurs when the brain is deprived of blood flow. One of the most common causes of stroke results from migration of material in blood vessels into the brain. An embolus, for example, often arises from the heart and lodges in the brain vessels. Blood flow may be reduced or totally obstructed as material travels into branch vessels and disrupts the arrival of oxygen and important nutrients to the brain. Stroke is often defined as occurring when brain dysfunction lasts for more than 24 hours. When the dysfunction recovers in less than 24 hours, this is referred to as a transient ischemic attack (TIA). Strokes and TIAs can involve any area of the brain. The resulting disability may be mild or lethal. Many patients are left with serious disabilities such as motor loss (e.g., inability to walk or use limbs), speech impairment, blindness, etc. In many elderly patients, recurrent episodes of microembolization can occur. In this situation, there is no clear stroke or TIA event, but the mental function of the patient slowly deteriorates. Also many elderly patients with extensive atherosclerotic disease in the aorta suffer from recurrent microembolization to the brain. The mental capability or status of these patients can slowly decay or worsen and may become so dysfunctional that these patients require placement in a nursing home.

The material that travels to the brain may vary considerably in composition. Embolic material may include blood clots and fragments of clot-like material (platelet emboli, fibrin, etc.), atherosclerotic debris, foreign materials introduced into the circulation, gaseous material, and infected debris such as occurs when a heart valve is infected. Any of these materials, or other materials, may travel into the brain and result in various levels of injury.

Emboli frequently arise from the heart. Clots may form inside the atrium or ventricle of the heart, such as when the heart beats irregularly or when the heart is dilated. An example of an irregular heartbeat is an arrhythmia termed atrial fibrillation, and an example of a dilated heart is one that is failing due to congestive heart failure. Clots can also form inside the heart when an aneurysm is present within the heart. Infection on heart valves, referred to as endocarditis, can produce a mixture of clot material and infectious organisms. This material can break off of the valve and enter the blood circulation. Since the blood flow to the brain is high, the final destination of these emboli is frequently the brain.

Atrial fibrillation is the single most common cause of stroke. It is estimated that one third of all ischemic strokes are due to emboli from atrial fibrillation. For people older than 60, the prevalence of atrial fibrillation is about 4% and for those individuals older than 80, the prevalence approaches 10%. With the aging of the population, it is estimated that the incidence of stroke will rise considerably over the next 20 years. Patients with atrial fibrillation who receive no treatment have a risk of stroke of 8% each year. This risk may be reduced by taking anticoagulant medication, but these medications carry certain risks, such as the risk of bleeding, and not all patients are able to tolerate such medication.

Material that originates anywhere in the body, such as blood clots in the legs, can also travel through the heart. For example, there may be a defect in the heart at the atrial or ventricular level, and material such as a clot from the leg, may pass through the heart and enter the systemic circulation (i.e., a paradoxical embolus), also resulting in a brain embolus.

Another source of emboli to the brain is the ascending aorta. Various patients, most notably elderly patients, develop serious atherosclerotic disease in the aorta beyond the heart. Small fragments of this material may dislodge and embolize. Also, clots may form on this atherosclerotic material and embolize.

One of the high risk time periods for embolization occurs during diagnostic or therapeutic procedures when devices are inserted inside the heart and great vessels. The devices, such as catheters, may dislodge material or even portions of the heart or great vessels, resulting in stroke or TIA.

Therapy to prevent stroke from embolization is aimed at reducing the sources of emboli. Anticoagulants such as warfarin or Coumadin® are frequently administered to reduce clotting. Various antiplatelet drugs may also be administered to the patient. If infection plays a role, antibiotic therapy may be appropriate and surgery may be necessary if a heart valve has a serious infection. To prevent paradoxic emboli from traveling from the brain, the patient may require various therapies to close atrial or ventricular defects.

Unfortunately, strokes, TIAs and brain deterioration are still quite frequent for patients on known treatments. Many patients suffer complications from anticoagulants or are not candidates for anticoagulation drugs because of their high risk for bleeding.

Ultrasonic energy consists of sound waves having frequencies above a level audible to the human ear, or about 18,000 Hz. There are two main classes of ultrasound presently utilized in medical procedures. Low power ultrasound having a high frequency in a range of about 5-7 MHz is used for diagnostic procedures, such as imaging, and high power ultrasound having a low frequency in a range of about 20 to 45 kHz has been used in therapeutic medical procedures. Certain methods and apparatus are known or proposed for using ultrasound energy to divert material in the circulation from traveling to the brain of a patient. For example, see U.S. Pat. No. 6,953,438 and PCT Publication WO 2005/076729. The devices and methods proposed thus far, however, have various shortcomings. For example, the proposals include the use of exterior collars and pads, and placing devices in the esophagus and trachea of the patient for delivering ultrasonic energy. With respect to exterior devices, aiming the ultrasound and power requirements are significant issues that will impact the effectiveness of material deflection in the aorta. Such exterior devices will be distant from the aorta and the angle to the aorta will vary. With respect to the proposed interior devices, the locations of the esophagus and trachea are behind the ascending aorta. Therefore, ultrasonic energy delivered from the esophagus or trachea could instead make the situation worse by failing to deflect material in the blood flow posteriorly in the ascending aorta and away from the origins of the great vessels leading to the head of the patient. Instead, the material could be deflected closer to the origins of the great vessels. Another proposal is to position ultrasonic transducers along the outside of the aorta. However, this is undesirable as it involves an open surgical procedure and would not be practical for use in providing stroke prevention during minimally invasive procedures.

It would therefore be desirable to provide apparatus, systems and methods utilizing ultrasonic energy and/or other mechanisms in various advantageous manners to address issues and concerns with the currently utilized and presently proposed treatments.

### SUMMARY

The innominate vein returns blood to the heart from the upper left extremity and the left side of the head region. This vein joins the superior vena cava on the right side just above the heart. As it crosses from the left side to the right side of the chest, the innominate vein overlies the take offs or origins of the three great vessels to the head. Thus, a device residing in a vessel, such as the innominate vein, in this general area may be outfitted with one or more ultrasonic energy emitters or transducers that may be directed toward the aorta and produce a wave or waves of energy that deviate(s) embolic material away from the origins of these brain vessels.

For example, it is a relatively routine procedure to introduce a catheter into the innominate vein. This may be accomplished as a short term or temporary procedure to protect the brain during an invasive procedure, such as a procedure on the heart or a blood vessel, or during the time that a patient may be at high risk for stroke. In this latter regard, this may be after a recent stroke or TIA or in many other high risk situations where there may be mobile clot in the heart or infected material in a patient with endocarditis. A catheter carrying one or more ultrasound emitters may be advanced via a needle puncture into the left subclavian vein and directed into the innominate vein, superior vena cava, and right side of the heart. Alternatively, any other access point to this location may be used instead, such as from the femoral vein into the inferior vena cava to the right side of the heart and superior vena cava. Another access point is the heart or superior vena cava which may be particularly useful during surgical or catheter procedures on the heart. A controller may then power and direct the output of this ultrasonic emitter device. An ultrasonic brain protection emitter can be coupled, integrated or otherwise used with one or more additional devices (such as devices for aortic valve procedures or any other procedure during which emboli may be produced) to prevent brain injury. Additional examples of devices that may be used in conjunction with the ultrasonic brain protection contemplated herein include: septal defect repair devices, heart valve repair/replacement devices, electrophysiological devices such as arrhythmia diagnostic or therapeutic devices, devices for blood procedures, devices for blood pump installation procedures, heart catheters, aortic catheters or any other devices used during cardiovascular procedures.

A chronic or long term version of this device may also be provided. In this type of embodiment, for example, a procedure may be performed very much like pacemaker insertion procedures. A pacemaker-like lead that carries one or more ultrasonic emitters may be inserted into the veneous system, such as the subclavian vein which then becomes the innominate vein before entering the superior vena cava and the heart. The ultrasound emitters may be attached to a controller and power supply and either or both of these components may be implanted in the body or external to the body. Alternatively, the controller and power supply may each be used outside the body. All components of the system may be portable in the sense that the patient may be mobile with all components carried in or on their body. In the case of using multiple ultrasound emitters or transducers, it may be advantageous to cycle the actuation of these emitters to help avoid wear and overheating, for example. In other words, the ultrasound emitters may be turned on and off or may be cycled to lower power levels at different times in order to achieve this result.

A controller and energy supply may be permanently implanted into the left shoulder area of the patient, for example, as is a standard pacemaker generator. Most likely, such a device would drain the power from a battery relatively quickly and, therefore, it may be desirable to provide a recharging device by percutaneous means, such as through TETS coils or by refueling. Refueling could be accomplished through the percutaneous addition of a fuel such as alcohol by using an intermittent needle puncture or by placing a small tube or catheter that traverses the skin and allows re-fueling of the device, or by other methods. Various implantable fuel cells may also be developed/used for powering one or more portions of the system.

To maximize the effectiveness of the ultrasonic energy, it may be useful to bias the catheter carrying the one or more emitters or transducers such that the emitters or transducers reside closer to the aorta and arch vessels. This may be accomplished by a number of manners, such as through the use of spring members or other resilient or biasing elements that position the transmitter or emitters as desired. The transducers or emitters may also be mounted on or held in position by a stent-like device or a tissue graft, such as by trapping the emitters or transducers between the stent-like device and the vessel wall, or otherwise carrying the emitters on a stent-like device or tissue graft.

The tip of an insertion catheter may be attached inside the heart, such as inside the right atrium and/or the right ventricle. Such an attachment may be performed with a standard method that is used with pacemakers, such as through the use of a small screw in a lead cable or wire. Anchoring the catheter or lead in the heart may then allow the lead to be tensioned against the wall of the innominate vein to reduce the distance to the aorta. This can avoid the need for stents or other elements in the innominate vein. Also, the anchor in the heart would allow for the lead to be rotated to a fixed orientation toward the aorta without other retaining elements and thereby more precisely direct the ultrasonic waves in the desired direction to more effectively divert material in the aorta from the great vessels.

It may also be desirable and useful to rotate the catheter so that the ultrasonic waves are directed toward the path of blood flow. This may be accomplished by biasing the ultrasonic transducers and then rotating the catheter to maximize the effectiveness of the treatment. The position or positions of the ultrasonic transducers or emitters can then be visualized by X-ray to ensure that the direction of the waves has been adjusted correctly. The ultrasonic waves may also be focused to produce optimal treatment.

It would also be possible to confirm the ideal orientation of the one or more ultrasonic transducers or emitters by placing one or more ultrasonic sensors mounted on a catheter temporarily or permanently placed inside the aorta near the great vessels. Using signals from the sensor(s), the one or more ultrasonic transducers or emitters may then be positioned and rotated inside the innominate vein and the superior vena cava to optimize their position and orientation and ensure the proper amount and orientation of ultrasonic energy into the blood flow path.

It may also be desirable to place one or more ultrasonic emitters or transducers into the superior vena cava or even into the right side of the heart as this may begin to move embolic debris away from the arch vessels earlier in the blood flow path. A series of ultrasonic emitters could be used to move particles or material away from the origins of the great vessels to the brain to avoid embolization. By sequentially deviating the particles, rather than merely forcing all particles or material to the inside of the aortic arch with a large single ultrasonic energy emitter, the particles or material may be moved with lower energy requirements and more certainty as the emitters may be strategically placed near the orifices or origins of the great vessels to the head. Such a strategy would reduce the risk of embolization and may also increase the energy efficiency of the system. A series of ultrasonic energy emitters may provide the best protection and such emitters, for example, may be located anywhere inside the right atrium, the superior vena cava, and/or the innominate vein. The location of each emitter and the power output of each emitter could be adjusted to produce the optimal ultrasonic pattern for brain protection.

In another aspect, the ultrasonic energy emitters may be placed inside a sheath. The sheath could remain in place and the emitters could be changed and repositioned inside the sheath if failure occurred or if there was a need to relocate the position of the emitters. The emitters could be contained in a replaceable pouch or other containment system and removed and replaced through a relatively routine removal and replacement procedure while leaving the implanted sheath or catheter in the patient. In this regard, the effect of the ultrasonic energy emitters may decay or lessen over time. By placing the ultrasound emitters within a long sheath inside the patient that is connected through the skin to the exterior, one or more components of the ultrasonic emission system may be removed and replaced by a simple, nonsurgical procedure. If the ultrasound system was contained in a sheath in a fully implanted system, replacement of one or more components of the ultrasonic energy emission system could still be facilitated by placement into a sheath, however, a small incision would be necessary to facilitate replacement.

In another aspect, it may be useful and desirable to add various sensors to the ultrasonic emission system. Such sensors may, for example, measure EKG, temperature, oxygen saturation, blood flow and/or other parameters. The sensors may be added to the catheter to provide additional information to the system and/or to doctors monitoring the system. Furthermore, ultrasonic emission strength (such as intensity or on/off status) could be timed to the heart rhythm of the patient as indicated by one of the sensors. Also, the ultrasonic transducers or emitters may generate undesirable amounts of heat and, therefore, it may be desirable or necessary to reduce the intensity of ultrasonic energy emission upon sensing an elevation in the patient's local or core temperature.

In another aspect, a cooling system or device may be provided in the blood flow, with or without an ultrasonic emitter or transducer. Cooling the blood flow to the brain can reduce the core temperature of the patient. Also, the temperature of the blood flowing to the brain may be reduced. When blood flow to the brain is impaired, brain tissue deteriorates quickly. Cooling the brain, even by a few degrees, lowers cerebral metabolism and reduces the severity of brain injury. In some patients, the entire circulation to the brain is turned off for prolonged periods, such as an hour or more, by cooling the body and the brain. Cooling may be achieved by circulating a cool fluid or gas through a catheter. Also, solid state cooling using the Peltier principle may be used, or a vortex tube device may be used to provide a cooled gas to a closed circulation system in a catheter.

Such cooling systems may also be used to cool the catheter-based ultrasonic energy emission system. This is in addition to the cooling that may be provided by the flow of blood itself. Since 40% of the blood flow in a human body passes through the superior vena cava, cooling by convection would be highly effective. Additional cooling features may be added to increase the effectiveness of heat transfer including the addition of conductive materials and increasing a surface area of the implanted catheter system that contacts the blood flow.

In another aspect, it may be useful and desirable to increase the ultrasound emitting surface by compressing the ultrasound emitters and allowing them to expand once they are released. This would allow the emitters to be introduced into a small space and thereafter occupy a larger space inside the vessel.

It may also be most appropriate to place all the vessel protection around the base of only a limited number of the great vessels to the head. It would be possible to protect the brain in this manner and have a surgeon connect the carotid on one side of the patient to the carotid, or other inflow vessel, on the other side of the patient. For example, the left carotid artery could be joined to the right carotid artery in the neck for inflow and disconnected from flow from the aorta. There would then be no need to protect the left carotid using ultrasonic energy—only the innominate artery would require such protection. In this manner, or other manners, the unprotected vessel could be disconnected from the brain circulation and therefore, a combination of surgical diversion and ultrasonic brain protection could be used to prevent emboli.

In other embodiments, devices are provided for directing ultrasonic energy through the aorta of a patient proximate the origins of the great vessels and include an expandable element configured to be delivered into a blood vessel in an unexpanded state and then changed to an expanded state. The expanded state may fix the device into a position suitable for accurately directing the ultrasonic energy through the aorta to divert material in the blood flowing through the aorta. For this purpose, an ultrasonic energy emitter is carried by the expandable element. The ultrasonic energy emitter may also be moved into position by the expandable element.

In various embodiments, the ultrasonic energy emitters are placed within the aorta and direct energy through the aorta from this interior location. In other embodiments, the ultrasonic energy emitters are located outside the aorta, such as in other generally adjacent blood vessels, and direct ultrasonic energy through the aorta from this outside or exterior position.

Various embodiments include ultrasonic energy emitters that may be assembled and/or spring or bias into position within the blood circulation and ultrasonic energy emitters that can move relative to each other at least while they are being placed or inserted into the blood circulation.

Other embodiments include stent-like devices and grafts outfitted with at least one ultrasonic emitter. The stent-like device and/or graft may be implanted within the patient for long term ultrasonic energy emission associated with chronic conditions for long term brain protection.

Still further embodiments involve kits or systems that may include one or more therapeutic and/or diagnostic devices associated with ultrasonic emitters. For example, therapeutic or diagnostic devices, such as catheter-based interventional tools, may be used inside blood vessels, such as the aorta, during procedures. The ultrasonic energy emission within the aorta, for example, may be used during any cardiovascular procedure for stroke protection in the event that material is dislodged by the device(s) during the procedure. The ultrasonic emitter or emitters may be carried directly on the interventional tools used during the catheter-based procedure, or may be carried on separate catheters or catheter-based devices used during the procedure.

These and other features, objects and advantages of the invention will become more readily apparent to those of ordinary skill in the art upon review of the following detailed description, taken in conjunction with the accompanying drawings.

### BRIEF DESCRIPTION OF THE DRAWINGS

FIG. 1A is a partially sectioned perspective view of a human heart and various blood vessels connected therewith, and illustrating a first embodiment of an ultrasonic energy emitting catheter device entering in the left subclavian vein and extending toward the superior vena cava.

FIG. 1B is similar to FIG. 1A, but illustrates insertion of the ultrasonic energy emitting catheter into the superior vena cava and extending generally toward the innominate and left subclavian veins.

FIG. 2 illustrates another perspective, partially sectioned view of the human heart, and illustrates another embodiment of an ultrasonic energy emitting catheter having an anchor element for maintaining the desired position of the catheter.

FIG. 3 is a schematic representation of a patient and the heart of the patient, partially cross sectioned, and further illustrating a manner of introducing a catheter device.

FIG. 4 is similar to FIG. 3, but illustrates the further connection of the catheter device to a power supply and controller for operating the ultrasonic energy emitting catheter.

FIG. 5 is a view similar to FIG. 4, but illustrating another type of power supply having transcutaneous energy transmission capability.

FIG. 6 is a view similar to FIG. 5, but illustrating a fully enclosed system that may be completely implanted in a patient.

FIG. 7 is a fragmented, sectioned view of the heart illustrating both an ultrasonic energy emitting catheter and a further catheter capable of sensing or detecting ultrasonic energy.

FIG. 8 is another sectioned view of the heart showing a mechanical method for biasing the ultrasonic energy emitters against a blood vessel to maintain their position during energy emission.

FIG. 8A is a cross sectional view taken generally along line 8A-8A of FIG. 8.

FIG. 9 is a sectioned view of the heart illustrating another mechanical method for maintaining the position and location of the ultrasonic energy emitter or emitters.

FIG. 10 is a fragmented and sectioned view of the heart illustrating an ultrasonic energy emitting catheter and a further catheter capable of sensing or detecting ultrasonic energy.

FIG. 10A is a cross sectional view taken generally along line 10A-10A of FIG. 10.

FIG. 11 is a schematic view of a patient and a cross sectional view of the heart of the patient illustrating the insertion of the catheter devices shown in FIG. 10.

FIG. 12 is a view similar to FIG. 11, and further showing a cooling element in the right side of the heart.

FIG. 12A is a sectional view of the heart illustrating the system of FIG. 12, with the further catheter removed.

FIG. 12B is a cross sectional view taken generally along line 12B-12B of FIG. 12A.

FIG. 13 is a fragmented, sectioned view of the heart, and illustrating a cooling element or device in the innominate vein and superior vena cava.

FIG. 14 is a sectioned view of the heart illustrating another cooling unit used in conjunction with a catheter placed into the cardiovascular system, such as the left subclavian vein and innominate vein.

FIGS. 15A and 15B are enlarged cross sectional views of a catheter device being used to introduce ultrasound energy emitters into a blood vessel in a reduced diameter or profile and then expanded for more effective ultrasonic energy emission.

FIG. 15C is a cross sectional view taken along line 15C-15C of FIG. 15B.

FIGS. 16A, 16B and 16C sequentially illustrate the use of an ultrasonic energy emitting catheter having ultrasonic energy emitters that may be adjusted in spacing with respect to the length of the catheter.

FIG. 17A is a fragmented, sectional view of the heart including a stent-like device carrying ultrasonic energy emitters.

FIG. 17B illustrates the stent-like device of FIG. 17A in one representative unimplanted state or condition.

FIG. 18A is a perspective view of another embodiment of a stent-like device having ultrasonic energy emitters.

FIG. 18B is a perspective view of a blood vessel graft element or device having ultrasonic energy emitters carried by the graft.

FIG. 19A illustrates a patient and the heart of the patient in cross section, and further illustrating another access point into the cardiovascular system for an ultrasonic energy emitting catheter.

FIG. 19B is an enlarged view of the heart shown in FIG. 19A with the ultrasonic energy emitting catheter in place and extending through the left ventricle into the ascending aorta.

FIG. 19C is a view similar to FIG. 19B, but illustrating the placement of a replacement aortic valve in conjunction with the use of the ultrasonic energy emitting catheter.

FIG. 20 is a fragmented and sectioned view of the heart illustrating another access point for an ultrasonic energy emitting catheter, i.e., directly into the ascending aorta.

FIG. 21 is a schematic view of a patient and a sectioned view of the patient's heart, and illustrating an access point for the ultrasonic energy emitting catheter into the superior vena cava and innominate vein through an initial pathway within a femoral vein.

FIG. 22A is a schematic view of a patient and a sectional view of the heart of the patient, illustrating the initial introduction of an ultrasonic energy emitting catheter having a positioning or stabilizing element associated therewith.

FIG. 22B is an enlarged view of the sectioned heart and deployment of the positioning or stabilizing element in the form of an expanding element.

FIG. 22C is a view similar to FIG. 22B, but illustrating full deployment of the expandable element.

FIG. 22D is a view similar to FIG. 22C, but illustrating another embodiment of the expandable element.

FIG. 23 is a schematic view of a patient and a sectioned view of the heart of the patient, illustrating an access point for an ultrasonic energy emitting catheter through a femoral artery and into the ascending aorta.

FIG. 24 is an enlarged sectioned view of the heart illustrating an ultrasonic energy emitting catheter including segmented elements as ultrasonic energy emitters.

FIG. 25A is a fragmented view of a heart and sectioned view of the ascending aorta, illustrating the initial introduction of another embodiment of ultrasonic energy emitters.

FIG. 25B is similar to FIG. 25A, but illustrates further deployment of the ultrasonic energy emitters.

FIGS. 26A, 26B and 26C sequentially illustrate the deployment of ultrasonic energy emitting elements from a catheter shown in cross section, as further illustration of the embodiment shown in FIGS. 25A and 25B.

### DETAILED DESCRIPTION OF THE DRAWINGS

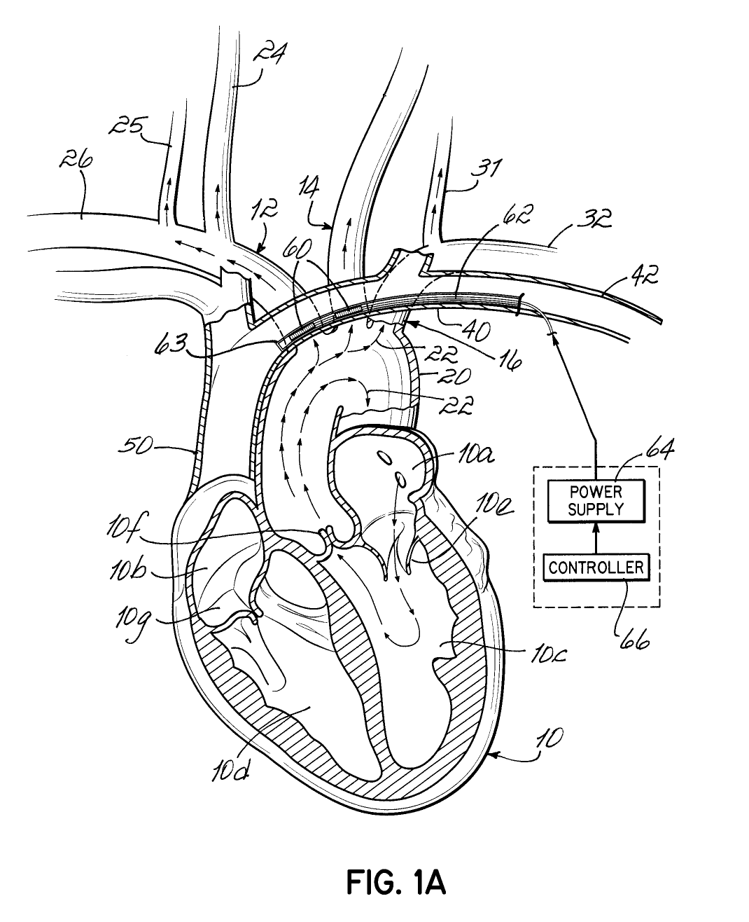

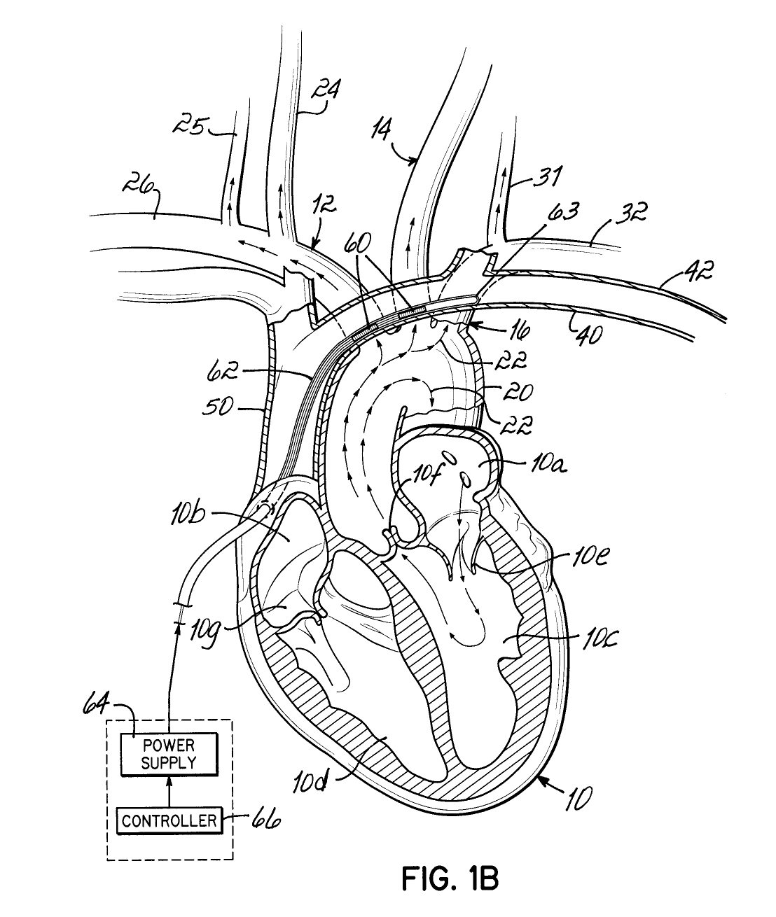

Like numerals in the various figures indicate like elements of structure and associated function. FIG. 1A illustrates a partial cross sectional view of the heart 10 and various connecting blood vessels. The heart 10 generally includes a left atrium 10a, right atrium 10b, left ventricle 10c, right ventricle 10d, mitral valve 10e, aortic valve 10f and tricuspid valve 10g. As shown, three great vessels 12, 14, 16 connect with the aorta 20 and receive blood flow as shown by the arrows 22. Specifically, these are the brachiocephalic trunk (or innominate artery) 12, the left carotid artery 14 and the left subclavian artery 16. The brachiocephalic artery or trunk 12 branches into the right carotid artery 24, the right vertebral artery 25 and the right subclavian artery 26. The left subclavian artery 16 gives rise to the left vertebral artery 31. Generally overlying these three branches 12, 14, 16 is the innominate vein 40, which starts as the left subclavian vein 42 and then joins the superior vena cava 50 as shown adjacent the aorta 20. A series of ultrasonic energy emitters 60 may be placed in the innominate vein 40 through the use of a suitable catheter device 62 inserted into another catheter or sheath 63. The ultrasonic emitters 60 are connected to an external power supply 64 and a control or controller 66. In each embodiment, power is controlled such that ultrasonic energy is delivered at appropriate therapeutic levels sufficient to divert or deflect material in the blood flow. As mentioned, the ultrasound may have a relatively low frequency of about 20 kHz to about 45 kHz. A particle or other material (not shown) may travel from or through the heart 10. If emitters 60 are not activated, this material may travel into one of the three great vessels 12, 14, 16 arising from the arch of the aorta 20, likely resulting in a stroke. While the ultrasonic emitters 60 are shown in the innominate vein 40, they may instead extend down the superior vena cava 50, adjacent to the aorta 20, and even into the right side of the heart 10 (which is on the left side of the drawing). Ultrasonic emitters could also be added to these locations to encourage the movement of material away from the arch vessels 12, 14, 16 earlier in the course of blood flow. This may improve reliability of material deflection and may reduce the power consumption. When the system is activated by supplying power to the emitters 60 from power supply 64, as regulated by the control or controller 66, the embolic material is deflected away from the great vessels 12, 14, 16. The embolic material then travels downwardly into the lower body through the aorta 20. The embolus is not necessarily eliminated but, rather, diverted to another organ or location in the lower body. In this regard, the legs, bowels, kidneys etc., are more tolerant of an obstruction.

FIG. 1B is similar to FIG. 1A and, as with other figures, like reference numerals in FIGS. 1A and 1B refer to like elements of structure and function and therefore need not be further discussed. FIG. 1B illustrates one of many other optional insertion or access points for the catheter device 62. As illustrated in FIG. 1B, this access point may be proximate to the right side of the heart and the superior vena cava 50 and, as shown in this illustrative embodiment, extending into the innominate vein 40. This places the ultrasonic energy emitters 60 generally in the same location as that shown in FIG. 1A for purposes as described above, and further purposes described below in connection with other examples.

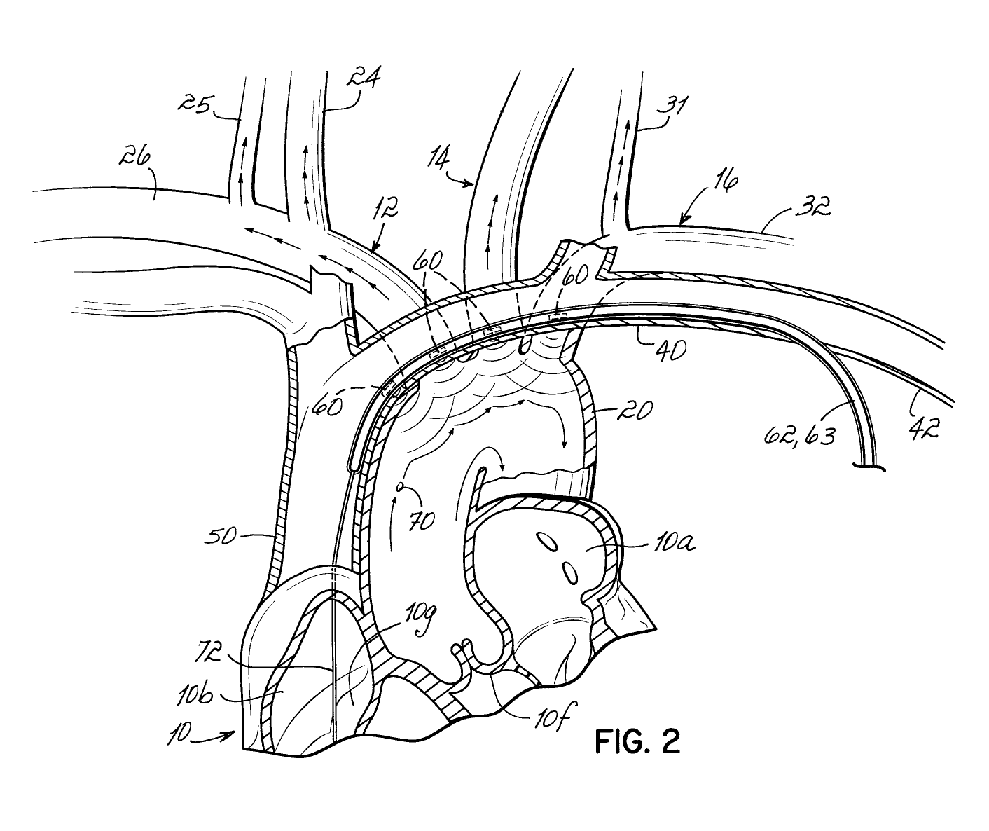

Referring to FIG. 2, using relatively high power, a particle or material may be deflected to the opposite side of the aorta 20, as illustrated by the sharply turned arrow on the inside of the aorta 20. One alternative is to limit the power consumption, heat generation and potential heat-related complications such as hyperthermia and blood clotting, by using a strategy of only deflecting the particles or material enough to bypass the brain vessels. Here, a series of emitters 60 sequentially deflect a particle 70 with a series of lower energy emissions. It will be appreciated that these emitters 60 may be cycled on and off and/or activated with different amounts of power in various cycles, to further reduce power consumption and heat generation. This may also increase the life of the system. As long as the material 70 is deflected adequately to bypass the last of the three arch vessels 12, 14, 16, stroke will be avoided. Additional deflection may not further reduce stroke risk but may make the device more complex and potentially increase risk or side effects.

The emitters 60 may also be placed in the superior vena cava 50 and right side of the heart 10 to move material 70 earlier in its path along the aorta 20. Anatomically, the superior vena cava 50 is located on the right side of the aorta 20. By beginning the movement of material 70 low in the aorta 20, the material 70 will already be biased to the inside of the turn in the aorta 20.

A plurality of emitters 60 allows for the development of strategies to variably adjust the power to each emitter 60 and the sequence of powering to optimize embolus (e.g., material 70) deviation or deflection based on the expected size of particles or material 70 in a patient. This also optimizes energy consumption for a given level of brain protection.

FIG. 2 also shows that the transducers or emitters 60 may be anchored to the heart, in a manner similar to a pacemaker, by inserting an anchoring lead 72 inside the right atrium 10b or right ventricle 10d (FIG. 1A) of the heart 10, for example. This may stabilize the location of the transducers or emitters 60 and, by tensioning the anchor cable or lead 72, the ultrasound energy may be moved closer to the aorta 20 to reduce the distance to the aorta 20 and reduce energy requirements. The ultrasonic energy emitting catheter 62 may have a larger number of ultrasonic elements 60 that may allow the system to emit ultrasonic energy beyond the innominate vein 40 such as through the superior vena cava 50 and the right side of the heart 10.

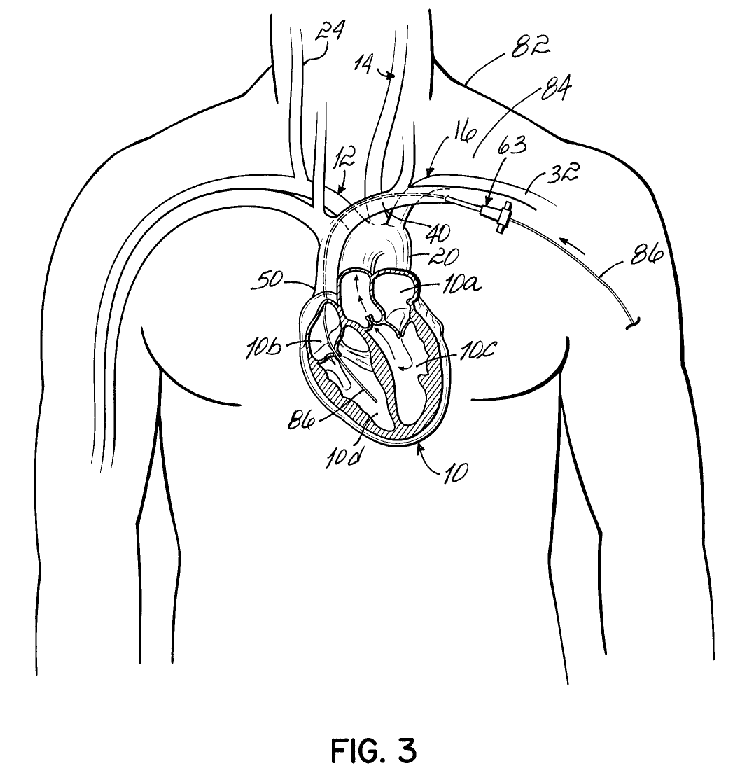

FIG. 3 shows one illustrative manner for introducing a catheter device 63 of the system into the patient 82. A needle puncture or a cut down is made in the shoulder region 84 of the patient 82 and a guidewire 86 is introduced into the subclavian vein 26 and further on into the right side of the heart 10. This guidewire 86 is then used to guide an ultrasonic device 62 into place, as will be described. An insertion catheter device 63 may include a channel (not shown) to allow the ultrasound energy delivery catheter 62 to pass over the guidewire 86. The channel may be along the entire length of the catheter 63, or only a portion of the catheter 63. It may also not be necessary to insert the ultrasound catheter 62 over a wire 86. Instead, the ultrasound catheter 62 may be inserted directly into the entry site in the shoulder area 84 of the patient 82. However, most doctors find it safer to use a guidewire 86 as there is less risk of passing the catheter 62 down an incorrect channel or perforating a vessel.

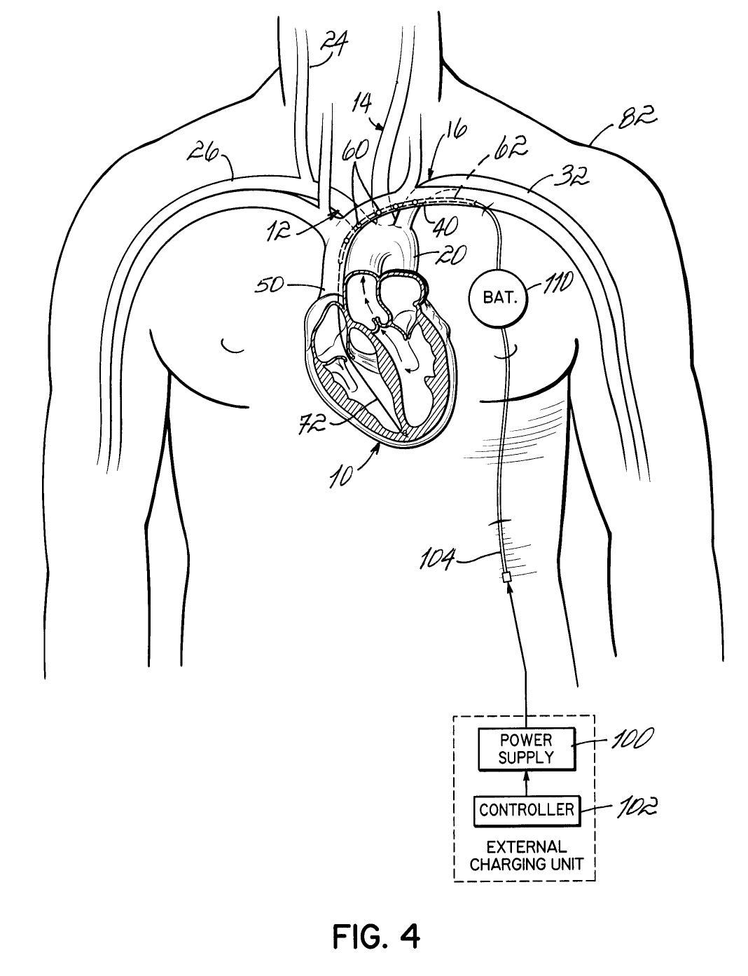

FIG. 4 illustrates the ultrasound emitter catheter 62 shown in position in the venous system. The emitter device or catheter 62 extends through the skin to the outside of the patient 82. The system may be powered by an external power supply 100 and regulated by a controller 102. Of course, the systems disclosed herein may have any suitable power supply and control, and these may or may not be integrated as a single unit. The electric cable 104 that extends outside the patient 82 would optimally be resistant to kinking, fracturing or other damage. Also, the cable 104 may require a covering with a material that promotes its attachment to tissue in the patient. This could include a variety of covers, but one typical cover is a fabric that has a velour-type surface or other characteristic that promotes tissue ingrowth. FIG. 4 also illustrates a system that includes an implanted battery 110. When the catheter 62 is disconnected from an external battery, the patient will risk a stroke during the time of disconnection. A battery 110 that is implanted may supply a limited amount of power to allow the patient to, for example, shower, change clothes, etc., without loss of protection. Note that although this is labeled a battery 110 in the drawing, a suitable control system would be coupled with the battery 110 to allow the ultrasound emitter device 62 to function.

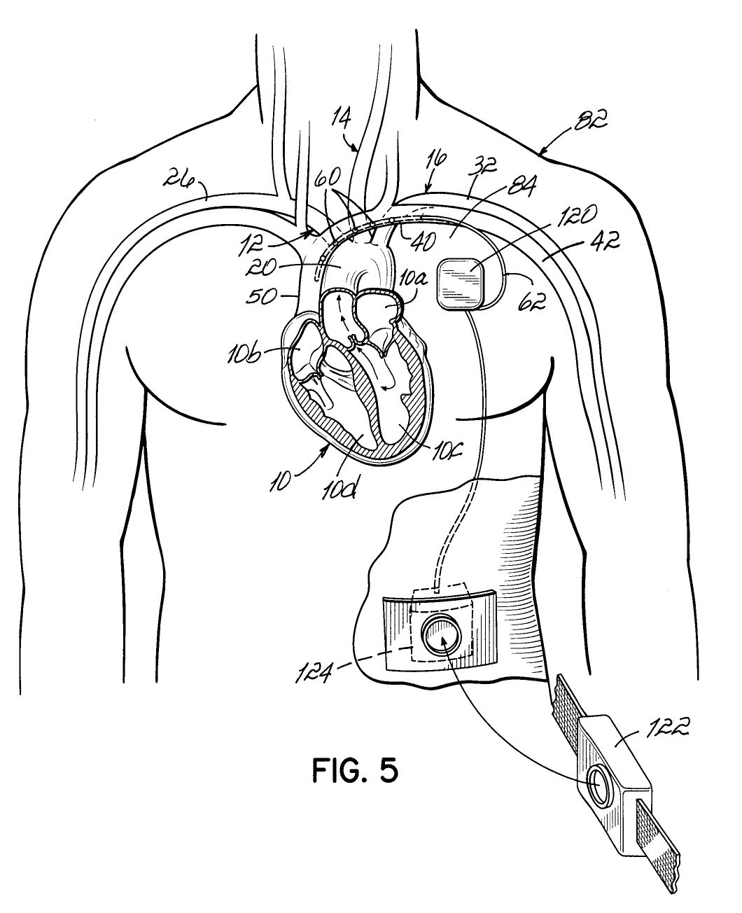

FIG. 5 illustrates a fully implanted system in accordance with another embodiment. This system does not require a cable or catheter extending from the patient 82. Instead, a battery 120 is implanted inside the patient 82. In this example, the battery 120 is shown implanted superficially in the shoulder or upper chest region 84 of the patient 82. In the abdominal region of the patient 82, a transcutaneous energy transmission system is shown. An energy emitter 122 is illustrated external to the patient 82, while an energy receiver 124 is implanted within the patient 82. These two components 122, 124 are aligned to optimize energy transmission. The receiver 124 is used to continually or intermittently recharge the battery 120 implanted in the patient 82. This arrangement is advantageous because there is no electric cable extending from the patient 82 and, therefore, mobility is easier. Also, a cable exiting the skin is a location for potential infection to enter the body and a closed system is therefore often preferred to prevent infection.

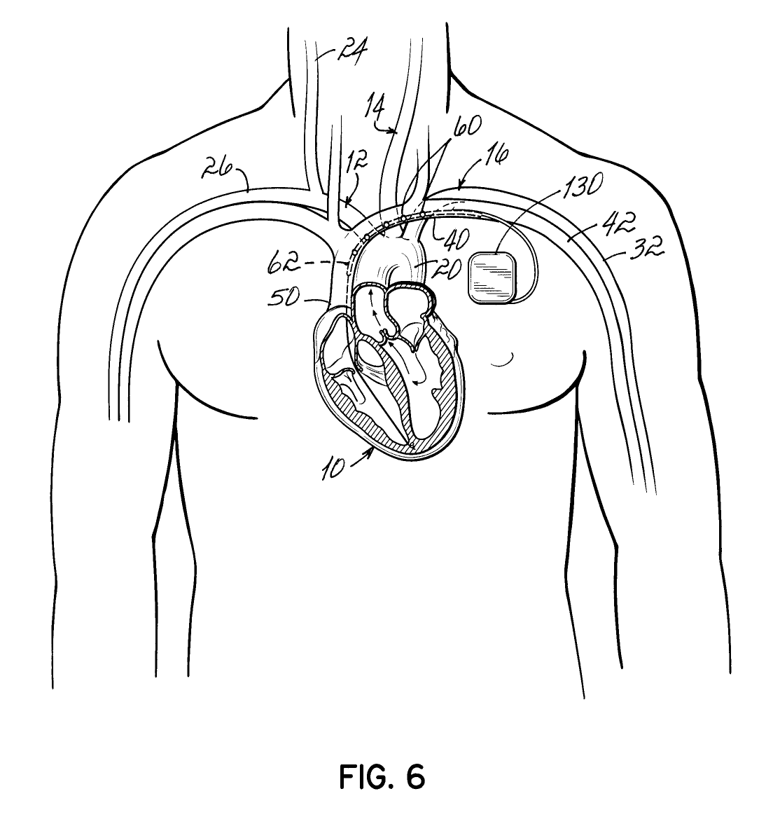

FIG. 6 illustrates a fully enclosed system that includes no external cable. This system may function adequately at low power, in a manner similar to a pacemaker. More likely, however, this system will require a fuel. For example, an implanted power generation unit 130 may be filled with a fuel by occasional injection of fuel into the system. Fuels are typically toxic but, with care, a fuel reservoir coupled and implanted with power generation unit 130 could be filled by needle injection. Instead of needle injection, a small catheter or catheters could exit the patient and this could be used to refuel the device and to remove waste products from the device if this were necessary. The waste products of a fuel cell may be water, carbon dioxide, or other material or elements which could be absorbed by the body and excreted by the kidney and the lungs or removed from the body if necessary or desired.

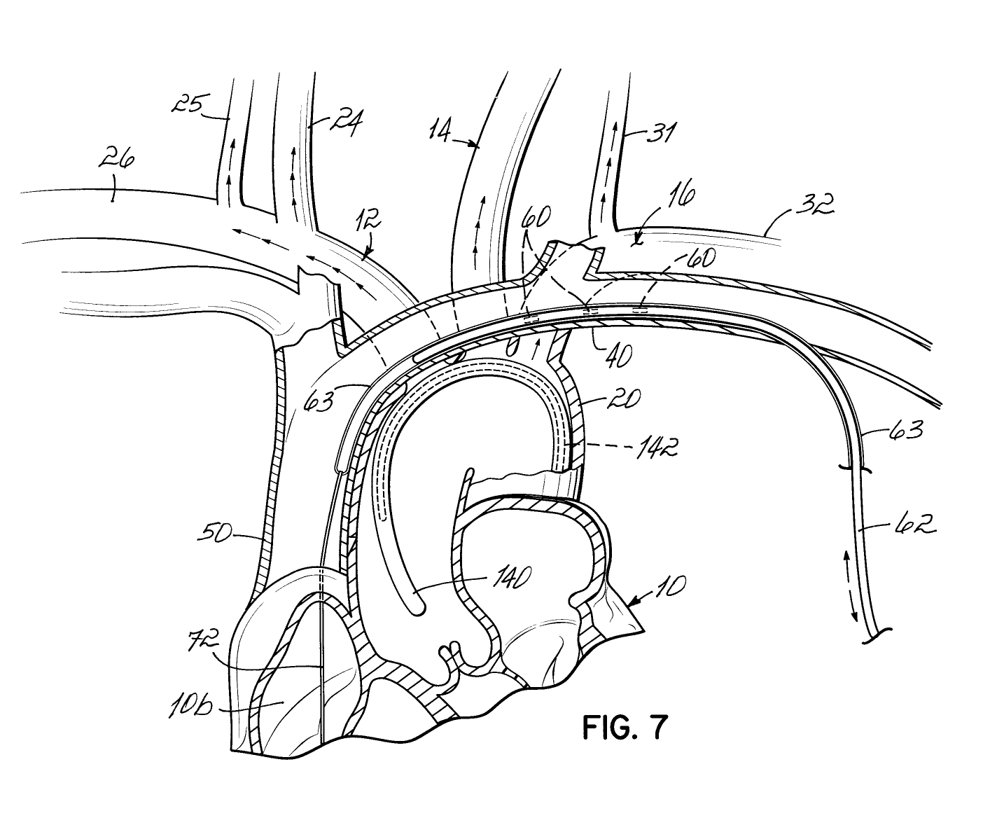

FIG. 7 illustrates that proximity of the ultrasound emitters 60 to the aorta 20 is important for reducing energy loss. Also, the implanted device 62 may be directable to more accurately aim the ultrasonic energy beam or waves. Thus, features for improving the consistency, accuracy and repeatability of the device position and/or orientation may be provided for increasing the chances of properly diverting the embolis material. The catheter 62 carrying the ultrasonic emitters or transducers 60 may be anchored inside the heart using the cable 72, as shown, and pulling on a catheter 63 that receives the transducer or emitter catheter 62 will move the assembly closer to the arch vessels 12, 14, 16. A separate catheter 140 may be inserted into the aorta 20 with a sensor or sensors 142 that detect the best orientation and position for the transducers or emitters 60. Such sensors 142 may, for example, be ultrasonic receivers that detect the strongest signal as the ultrasonic transducers or emitters 60 are positioned, rotated or otherwise oriented by the surgeon within the catheter or sheath 63 that is initially implanted.

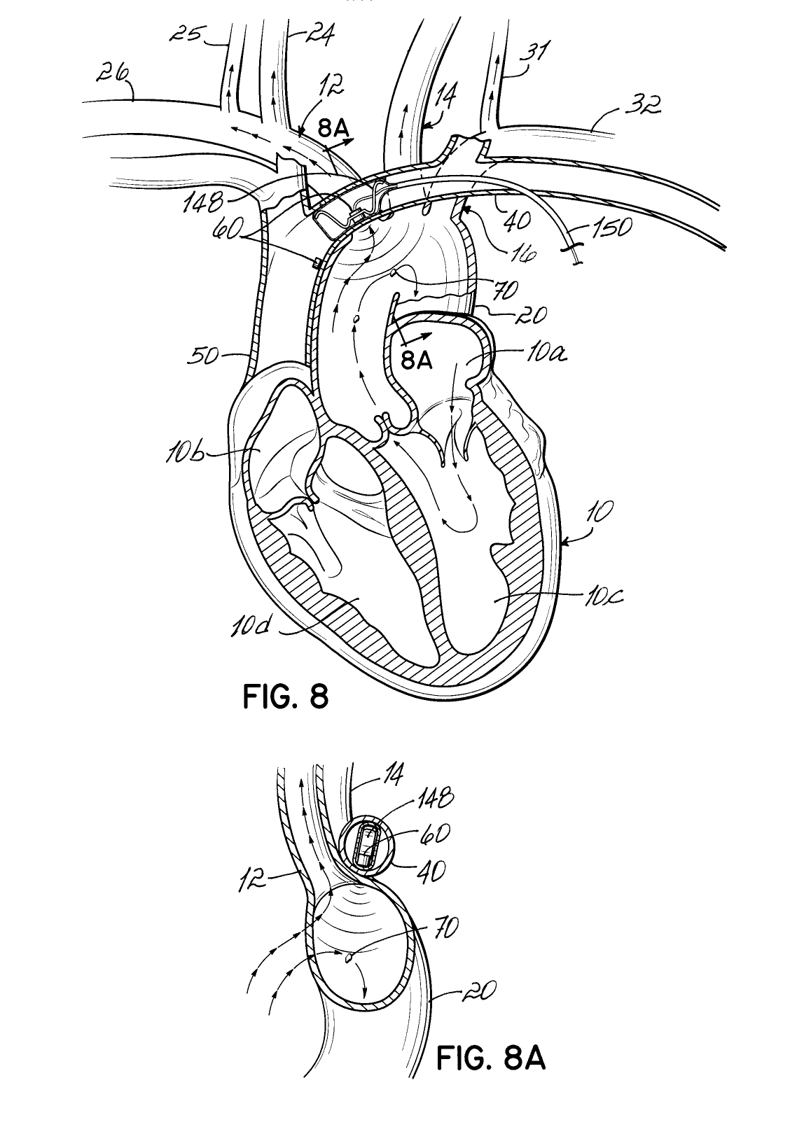

FIG. 8 illustrates a mechanical method for biasing the ultrasonic energy emitters 60 against the innominate vein 40 and maintaining their position, as shown, with a biasing member 148 (only one shown). FIG. 8A, which is a cross section taken along line 8A-8A of FIG. 8, illustrates the interior of the innominate vein 40 and the adjacent section of the aorta 20. This demonstrates that the transducer or transducers 60 are moved closer to the aorta 20 and great vessels 12, 14, 16 in a reliable location. Once the transducer or transducers 60 are in position, the transducer or transducers 60 may be released from the catheter 150 used for introduction and the catheter 150 may be removed.

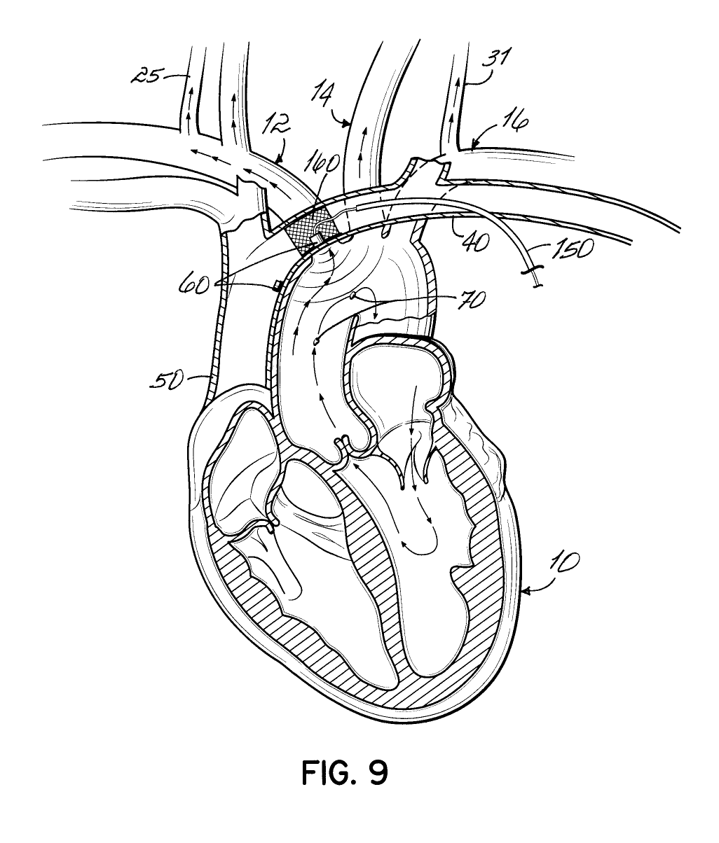

FIG. 9 illustrates that other mechanical means or methods may be used to position and maintain the location of the transducer or transducers 60. In this example, a stent-like device 160 is illustrated. This stent-like device 160 may be permanent or absorbable. In this latter regard, one possibility is that the transducer 60 may be held in position for a period of days to weeks, and the absorbable stent or supporting structure 160 may be absorbed or dissolved in the body and the transducer 60 would thereafter maintain its position due to scarring or tissue overgrowth.

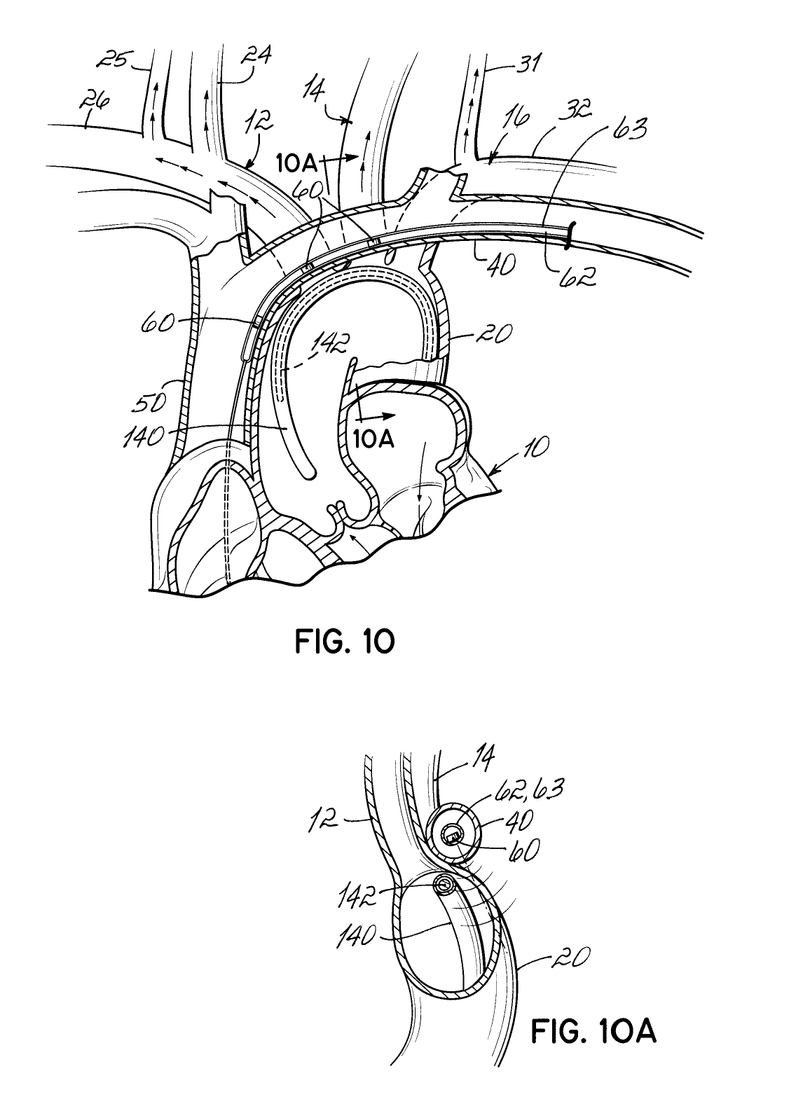

FIG. 10 is a view similar to FIG. 7. FIG. 10A is a cross section taken along line 10A-10A of FIG. 10. A series of ultrasonic emitters 60 are shown in the innominate vein 40 and a temporary ultrasonic energy detector or receiver 142 is placed inside the aorta 20 in the region of the great vessels 12, 14, 16. The ultrasonic energy emissions or waves may be focused, rotated and adjusted to optimize the delivery of energy. The emission pattern, intensity, focus, location and rotation may all be adjusted by the doctor when inserting the device 62, with the effect of the adjustments being determined by the receiver 142 inside the aorta 20. Such a receiver 142 is not necessary, but may be valuable tool to help reduce the level of power output of the device, especially during chronic or long-term use.

The catheter in the aorta could also be used for brain protection. The ultrasonic receiver(s) or detector(s)s could be replaced or supplemented with ultrasonic emitters. If this catheter is positioned near the origin of the great vessels and the emitters directed appropriately, any embolus could be re-directed away from the head vessels. Brain protection could also be outfitted on existing catheters such as coronary angiography catheters or any other catheter used in any procedure, including blood pumps (such as balloon pumps) and other catheters that are situated in the aorta or other nearby vessels. This may be desirable during procedures where emboli could be created by mechanical trauma such as heart catheterization, aortic catheterization, aortic valve procedures, or other diagnostic or therapeutic (e.g., open surgical or catheter-based interventional) procedures at various levels of invasiveness into the body. Ultrasonic energy emitters could be carried on any temporarily or permanently implantable device capable of being directed, positioned and/or implanted within the cardiovascular system of the patient.

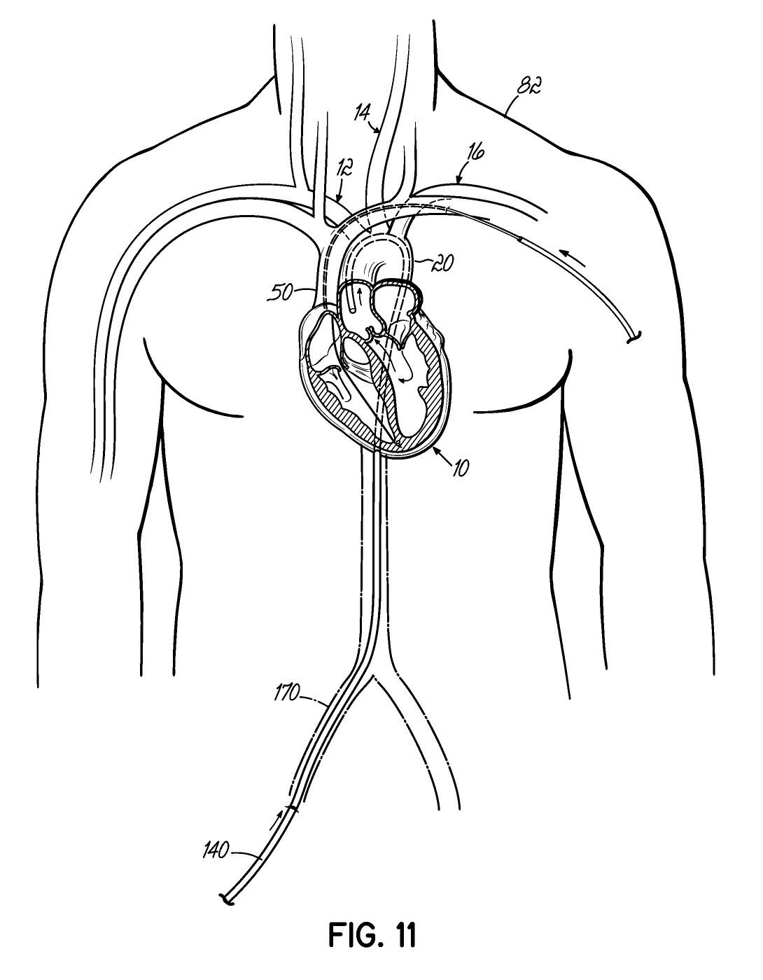

FIG. 11 demonstrates an ultrasonic receiver catheter 140 that is temporarily located in the aorta 20. This catheter 140 is inserted by a groin puncture into the femoral artery 170 (i.e., the typical location for a heart catheterization). As another example, the ultrasonic receiver catheter 140 may be threaded up an artery in the arm of the patient 82.

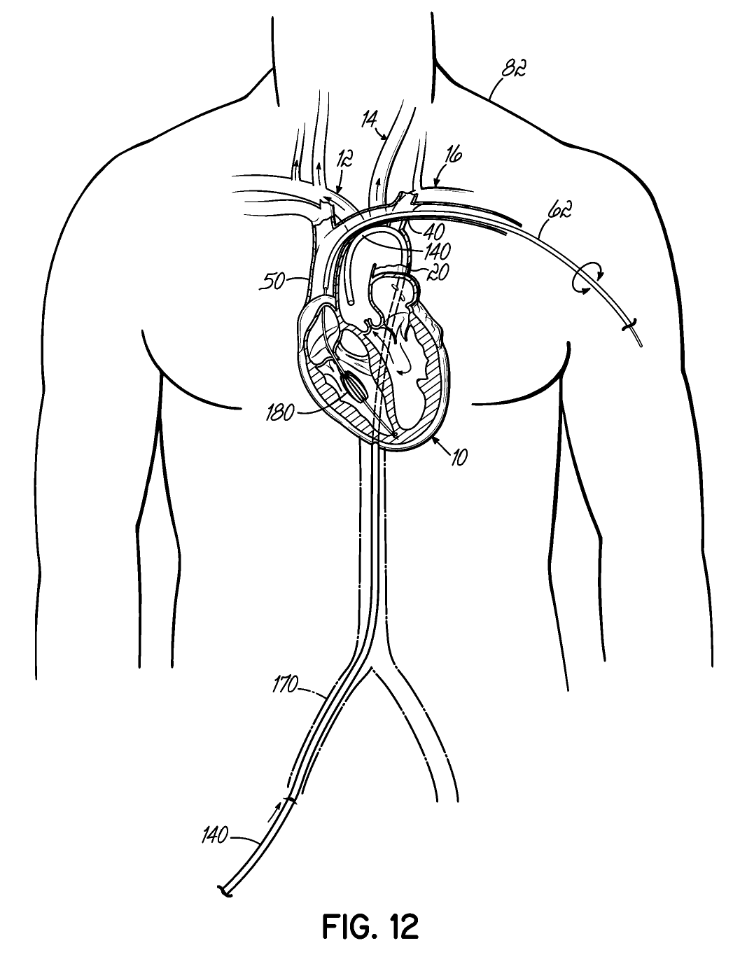

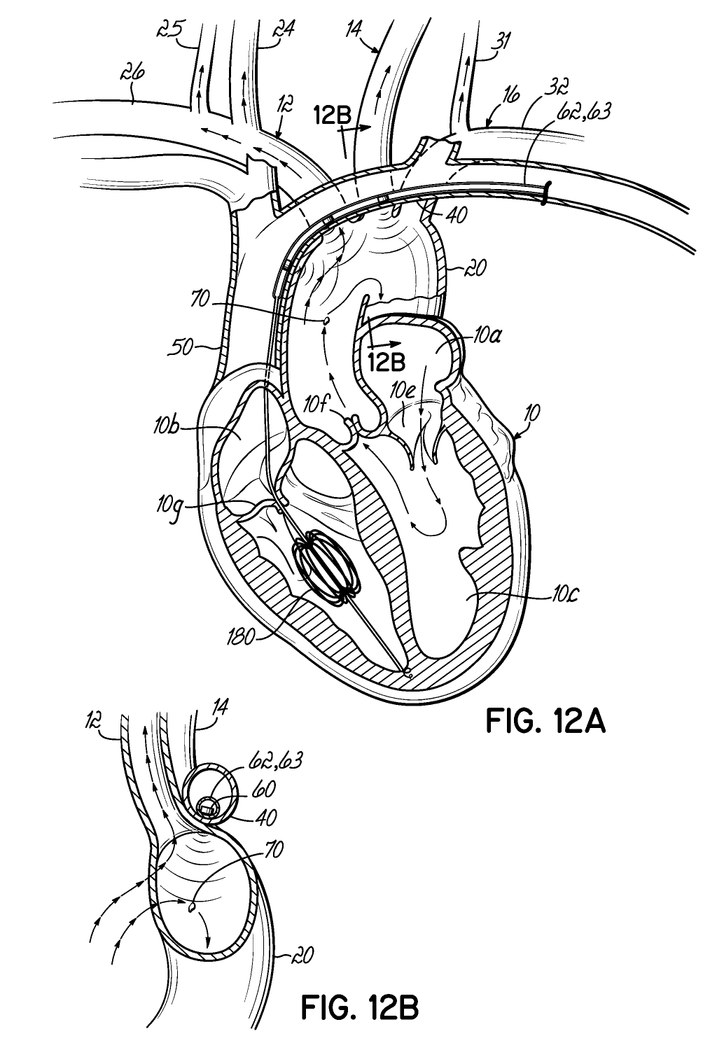

As illustrated in FIG. 12, an ultrasonic emitter catheter 62 is being rotated for adjustment purposes. The ultrasonic emitting catheter 62 may also be adjusted by movement in a forward/backward direction, and/or other adjustments. Control elements may be used to adjust the focus and intensity of the ultrasonic energy. An ultrasonic energy sensing or receiving catheter 140 is being used to assist in optimizing the rotation and final orientation of the emitter catheter 62. The ultrasonic receiver catheter 140 may also or instead be used as an ultrasonic emitter. By directing the ultrasonic energy to force material away from the aorta 20, the catheter 140 inside the aorta 20 may be used for brain protection in a manner similar to the ultrasonic emitter catheter 62. This intraaortic brain protection could also be used in conjunction with catheters and devices that currently reside inside the aorta 20 such as stents, stented valves, balloon pumps, heart pumps, etc. As an alternative or additional manner of orientation, the ultrasonic emitting catheter 62 and/or transducers or emitters 60 themselves may include radiopaque markers (not shown). A fluoroscopy unit (not shown) may be then used to determine the location, orientation and focus of the emitting catheter 62 and/or emitters 60. FIG. 12 further illustrates a basket-like element 180 in the right side of the heart. This may be a heat transfer element for carrying heat away from the blood flow and cooling this area of the anatomy to counteract any heating that is occurring due to the use of the emitters 60. Any suitable cooling device may be used, such as Peltier devices or other compact cooling units. FIGS. 12A and 12B illustrate a system similar to that shown in FIG. 12, but without illustrating the ultrasonic receiver catheter 140. FIG. 12B is a cross section taken along line 12B-12B of FIG. 12A. It will be appreciated that a smaller version of the cooling unit 180 shown in FIG. 12A may be used and, for example, reside in the superior vena cava 50 or even in the innominate vein 40.

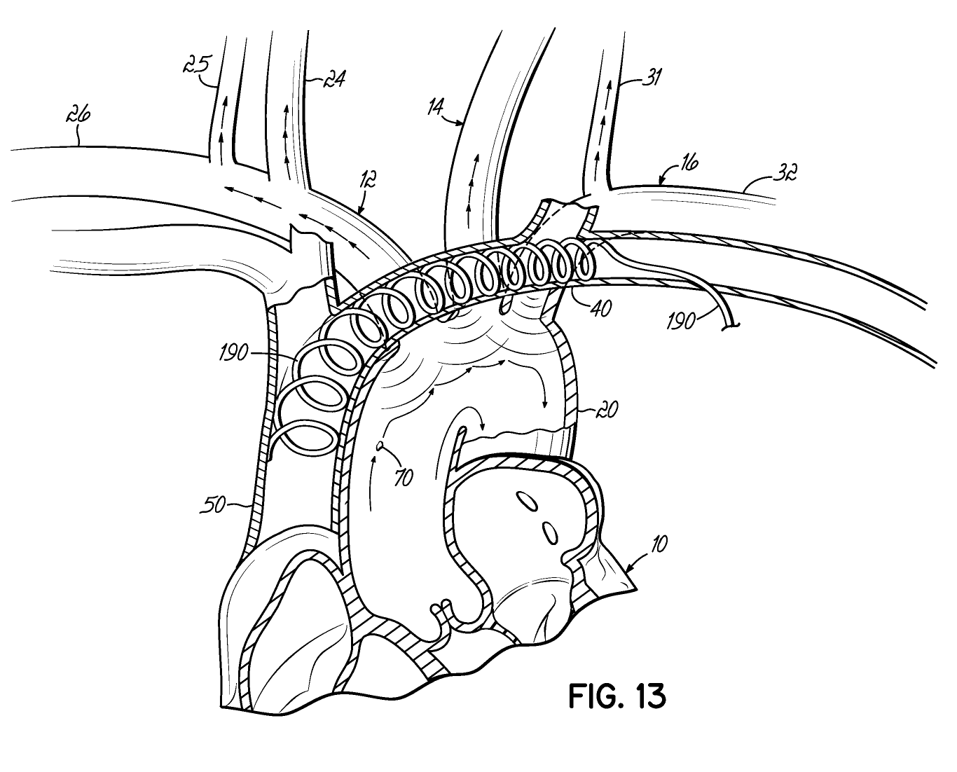

FIG. 13 illustrates another system that may increase the surface area for cooling. This system comprises a coiled catheter device 190 that may contain an inflow and an outflow so that cooled fluid (liquid or gas) may flow into the catheter 190, cool the ultrasonic catheter system (not shown) and the blood, then be returned out of the patient. In this regard, approximately five liters of blood flows through the heart 10 each minute. Conduction of heat may be accomplished with heat dissipation into the blood that flows by it. This concept is also shown in FIG. 12. Approximately 40% of the blood returning to the heart 10 flows down the superior vena cava 50. Therefore, the superior vena cava 50, as well as the innominate vein 40 may also be used for conductive cooling purposes.

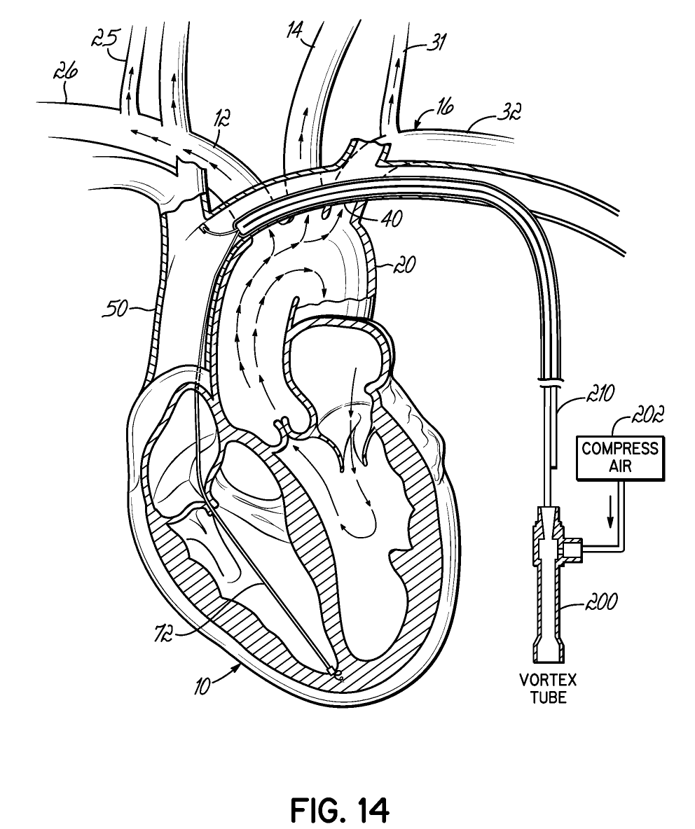

Another cooling unit is shown in FIG. 14 in the form of a vortex tube 200 that may be used for cooling purposes. As is generally known in the cooling arts, such vortex tube devices 200 use compressed air 202 to discharge air at a reduced temperature. This may be used with coils 190, such as shown in FIG. 13, or without coils. The air would not be directed into the blood stream but, instead, for example within an inflow and outflow tube 210 as schematically shown. It is important to note that brain cooling can be a significant factor in preventing damage when there is a reduction in blood flow to the brain. Brain vessels could be cooled by a system located inside the innominate vein 40, the superior vena cava 50 and/or the right side of the heart 10. Thus, brain protection may be achieved even without using the ultrasonic energy concepts disclosed herein, by using cooling elements implanted in one or more of these regions to reduce the temperature of blood flowing into the brain and by reducing the core temperature of the body. Any cooling method, such as Peltier/solid state, circulating fluid or cooled gas may be used to achieve this effect.

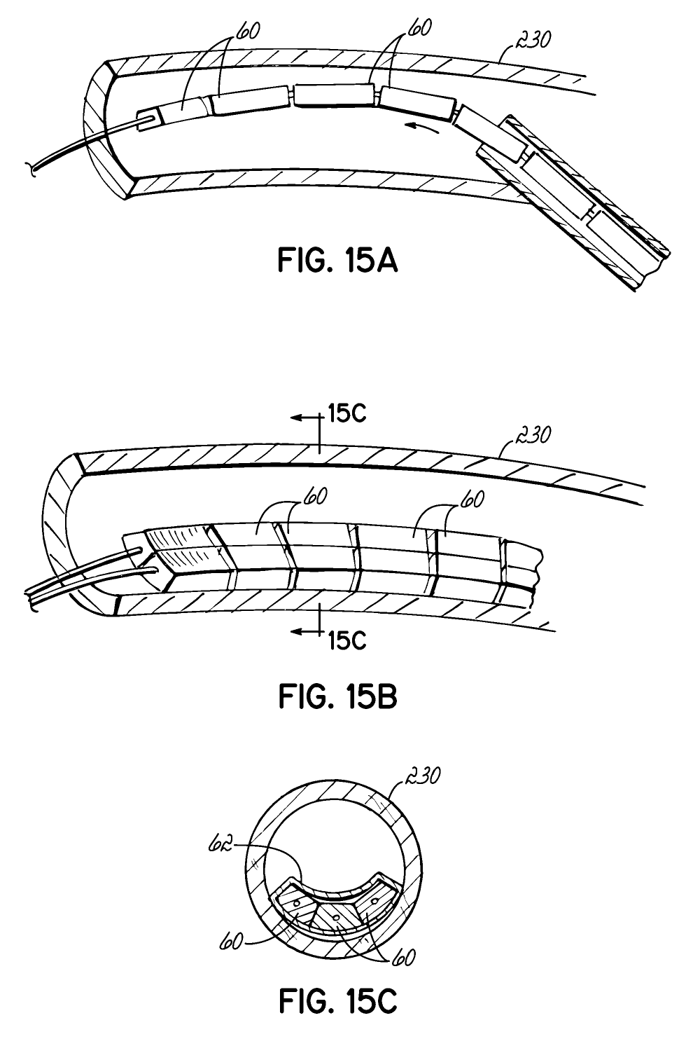

FIGS. 15A-15C illustrate the concept of introducing ultrasound energy emitters 60 in a reduced diameter or profile, for example, to allow easier insertion through a small incision or needle puncture, and then expanding the profile for more effective ultrasonic energy emission. One example of a reduced profile is shown in FIG. 15A, while the expanded profile is shown in FIGS. 15B and 15C, with FIG. 15C being a cross section taken along line 15C-15C of FIG. 15B. The emitter assembly 62 may be adjusted to fit the inner profile of the vein or vessel 230 in which it sits. As shown best in FIG. 15C, the emitters 60 may, for example, be aligned side-by-side in a lower profile, wider configuration within the vessel 230 when in use. The emitter assembly 62 may closely fit or follow the inner profile of the vessel 230 as shown in FIG. 15C or, on the other hand, may be turned 180° from the orientation shown in FIG. 15C to help focus the ultrasonic energy beam or waves.

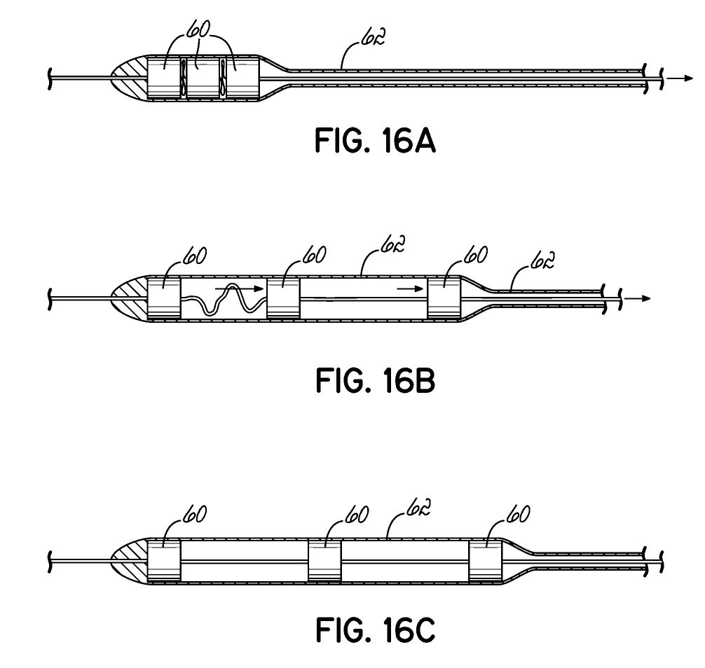

FIGS. 16A, 16B and 16C illustrate, in sequence, that the relative locations of ultrasonic energy emitters 60 may be adjusted within a catheter device or sleeve 62. These emitters 60 may be located closer together or farther apart depending on the needs of the particular patient and as decided by the doctor, for example.

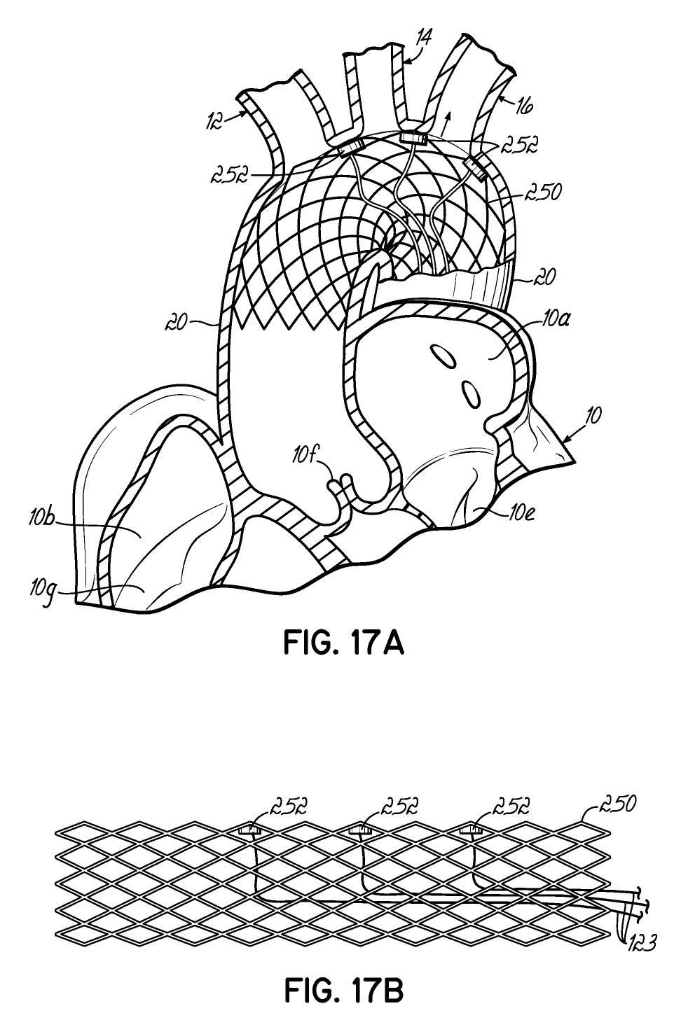

FIGS. 17A and 17B illustrate a stent-like device 250 which, in this embodiment, is a mesh material formed from, for example, wire without any fabric covering. The stent-like device 250 is shown implanted in the aorta 20 in FIG. 17A and is shown in its unimplanted state in FIGS. 17B. As the stent-like device 250 is made from an open mesh construction, blood may freely flow into the origins of the great vessels 12, 14, 16 as shown in FIG. 17A. The stent-like device 250 carries one or more ultrasonic emitters 252 which, when activated via wire leads 254 in accordance with any of the general principles discussed herein, divert material away from the origins of the great vessels 12, 14, 16 and downward through the turn in the aorta 20.

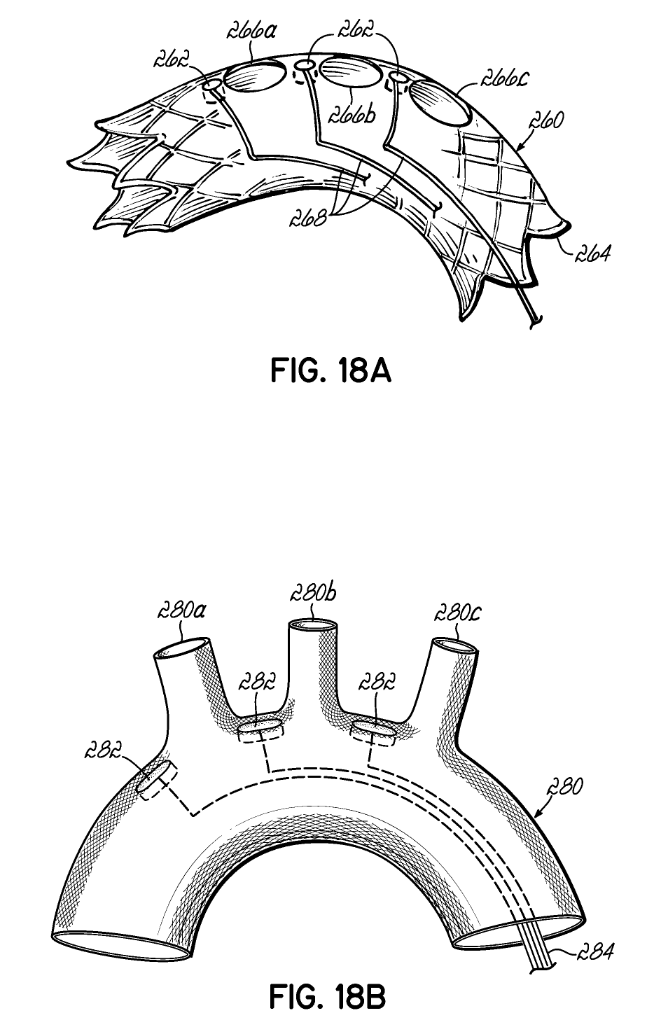

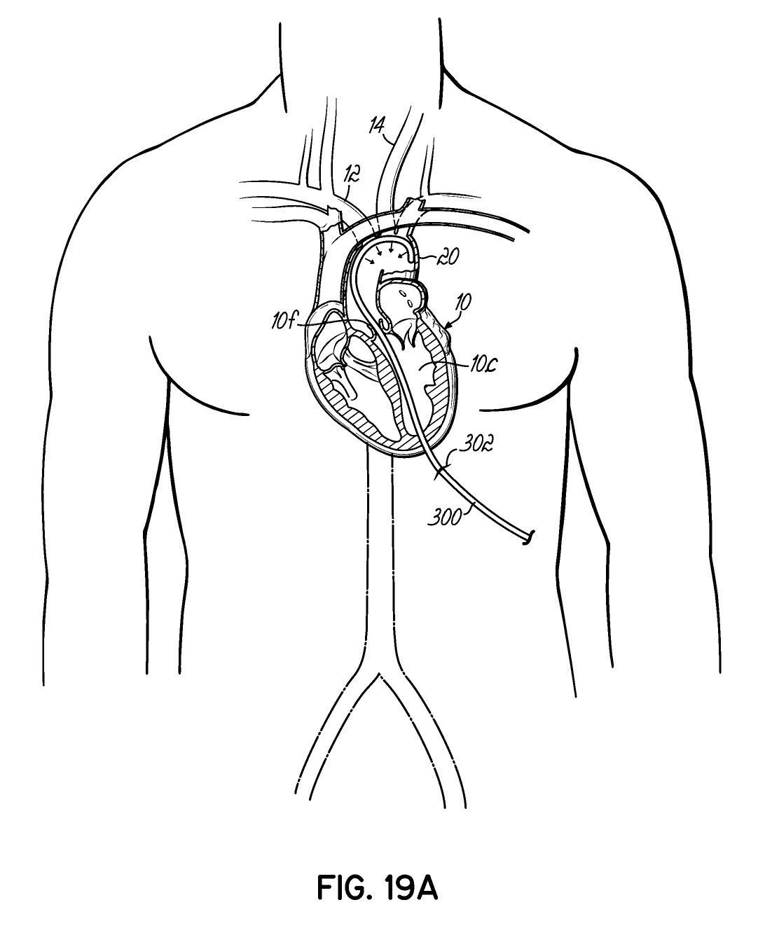

FIG. 18A illustrates another embodiment of a stent-like device 260 having one or more ultrasonic emitters 262 mounted thereto. This stent-like device 260 is also a mesh material, but is covered by a fabric 264 or other material which may promote the ingrowth of tissue. Openings 266a, 266b, 266c are provided in the fabric and the mesh, as necessary, to provide a blood pathway to the great vessels 12, 14, 16 (not shown). Suitable wire leads 268 may be used to power and control emitters 262. The emitters 262 and wire leads 268 may be located anywhere on the stent-like device (e.g., inside, outside or embedded) as desired, or as necessitated by the application.

FIG. 18B illustrates another embodiment in the form of an aortic graft 280. The aortic graft 280 can be formed of any suitable material and shape. In this example, it takes the form of the curved portion of the aorta 20 (FIG. 1A) with three graft segments 280a, 280b, 280c corresponding to the three great vessels. The graft 280 incorporates or carries ultrasonic emitters 282 coupled with one or more suitable wire leads 284 for control and power purposes as discussed herein. The aortic graft 280 may otherwise be formed according to known methods. Again, the emitters 282 and wire leads 284 may be located anywhere on the graft 280, as desired or as necessitated by the application.

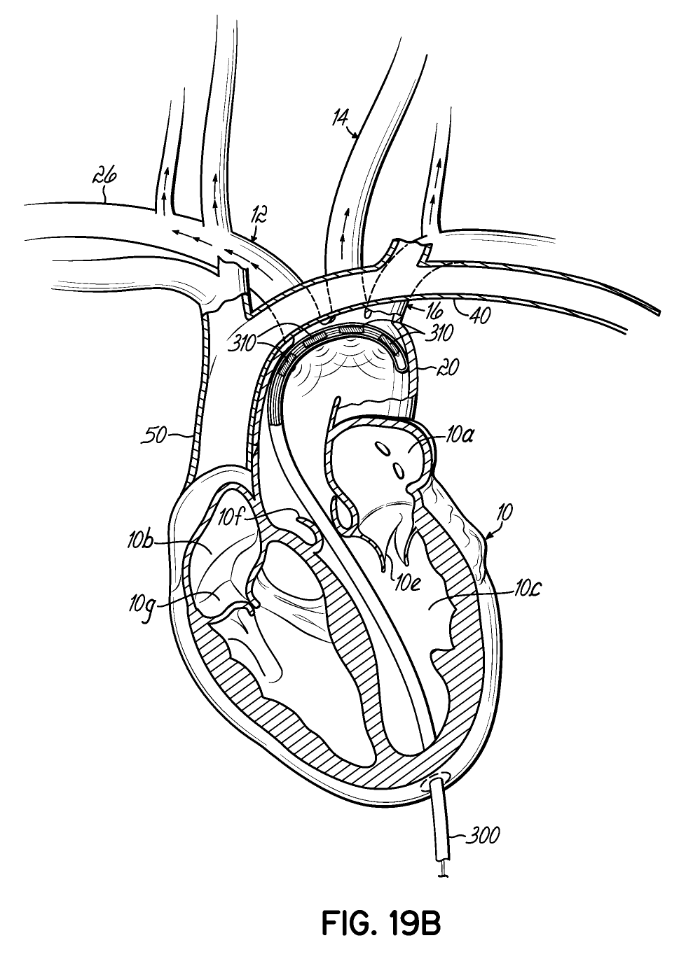

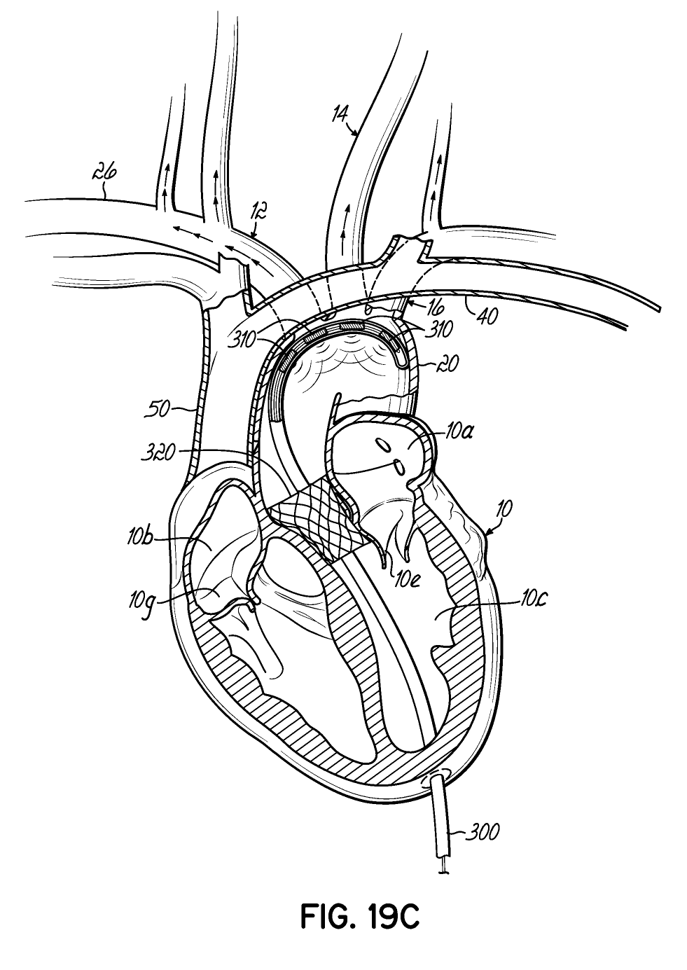

FIGS. 19A, 19B and 19C illustrate another potential access point or pathway into the heart 10 for a catheter 300 carrying one or more ultrasonic energy emitters. In this regard, the catheter 300 may be introduced through a small incision 302 in the chest area of the patient and directly inserted into the left ventricle 10c, through the aortic valve 10f, and into the aorta 20 as shown generally in FIG. 19A. As shown in FIG. 19B, the ultrasonic emitters 310 may then be used permanently, or more likely, temporarily to ultrasonically divert material in the blood flow away from the origins of the great vessels 12, 14, 16. Such a temporary installation of one or more emitters 310 may, for example, be desirable during any procedure in which embolism and stroke is more likely to occur. As shown in FIG. 19C, the catheter 300 may carry or be used to guide a stent 320 which, for example, mounts a replacement aortic valve, such as a mechanical or biological replacement valve. The ultrasound emitting catheter may be integrated into or separate from the catheters or catheter devices used for a procedure such as aortic valve replacement or any other therapeutic or diagnostic procedure. During such a valve replacement procedure, for example, there is a risk that material will break away from the native aortic valve and travel upwardly in the bloodstream through the aorta and into the great vessels 12, 14, 16. This embodiment would help prevent stroke by ultrasonically diverting such material downwardly through the turn in the aorta 20 at least while the patient is undergoing the associated therapeutic or diagnostic procedure, each of which may or may not be catheter-based procedures. One additional advantage of introducing an ultrasonic emission catheter into the aorta is that it can prevent the need for a separate veneous entry.

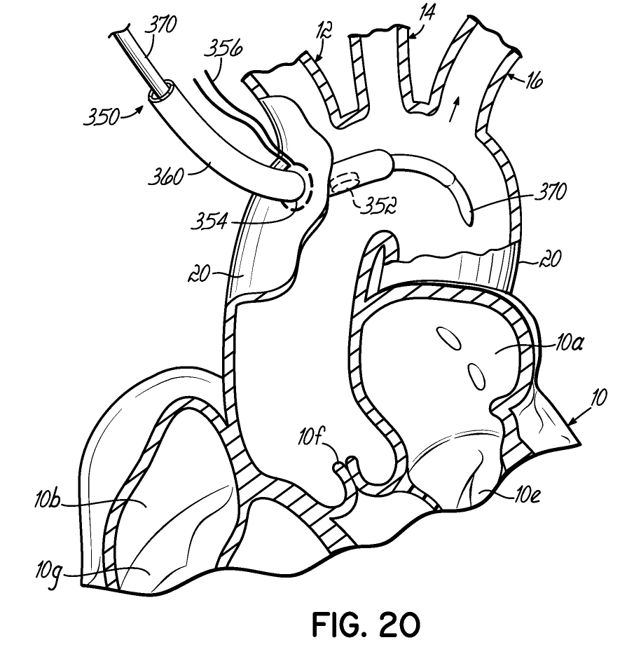

FIG. 20 illustrates another access point or pathway into the aorta 20 for a catheter device 350 carrying one or more ultrasonic emitters 352. In this regard, an incision 354 may be made directly in the aorta 20 and a purse string suture or sutures 356 may be used to secure a sheath 360 in the access point. The sheath 360 itself may carry one or more ultrasonic emitters 352, or a catheter 370 inserted through the sheath 360 may carry one or more ultrasonic emitters. The sheath 360 may also provide a pathway into the aorta 20 for one or more devices that may be used during a therapeutic or diagnostic procedure. The ultrasonic emitters 352 may then be activated during the procedure to divert any material in the blood flow away from the origins of the great vessels 12, 14, 16. This may, for example, be a temporary implantation of the ultrasonic emitters 352 used only during the procedure when the chances of stroke may be higher.

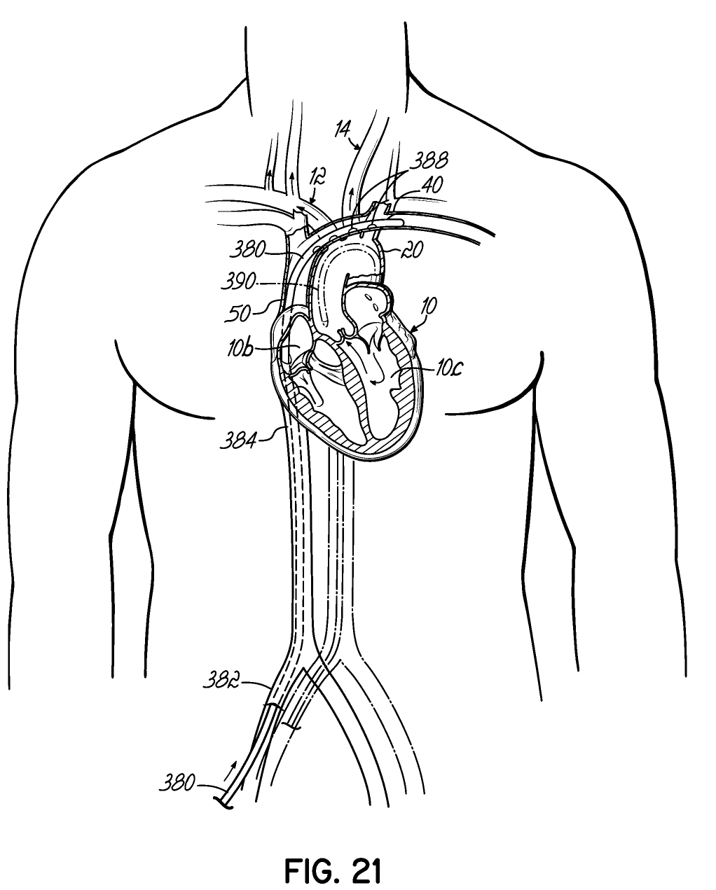

FIG. 21 illustrates another access point and pathway into the heart 10, superior vena cava 50 and innominate vein 40. In this regard, a catheter 380 is inserted in a femoral vein 382 and upwardly through the inferior vena cava 384, the right side of the heart 10 and into the superior vena cava 50 and innominate vein 40. This illustrates another pathway to the location(s) generally discussed above for providing ultrasonic energy emission from emitters 388 in one or more portions of the veneous system adjacent to the aorta 20. It may be very convenient to place the catheter in the venous system for embolic protection while a cardiologist or surgeon performs a procedure involving the heart or the arterial system. This would provide embolic protection without the interference of too many catheters introduced into the arterial system. As further shown in dash-dot lines, another catheter 390 may be placed within the aorta 20 itself for various purposes, such as ultrasonic sensing, therapeutic procedures, additional ultrasonic diversion of material, or other reasons. As mentioned previously, the ultrasound emission could be combined with other catheters used for various procedures inside the aorta or the heart, for example.

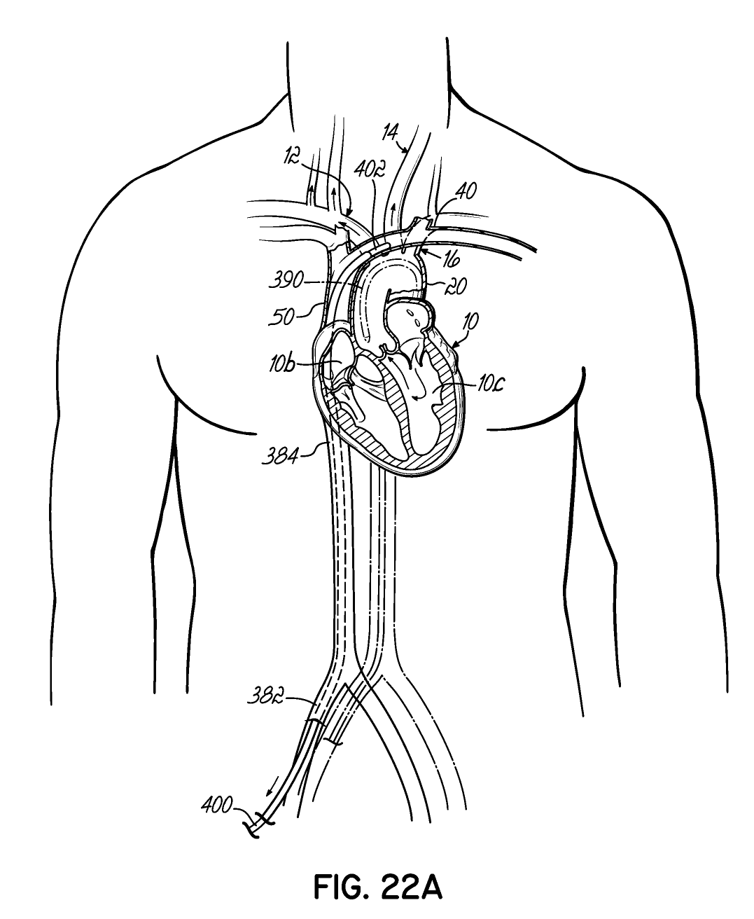

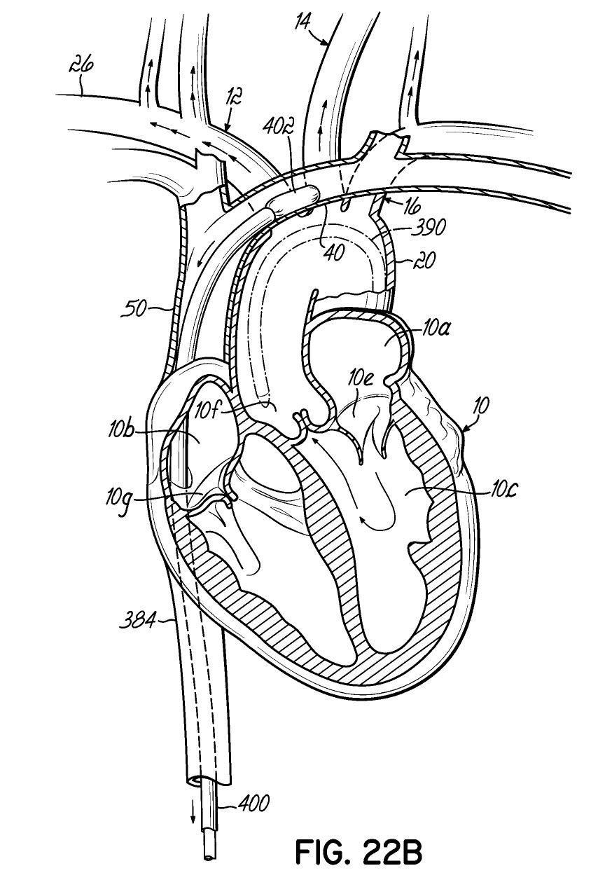

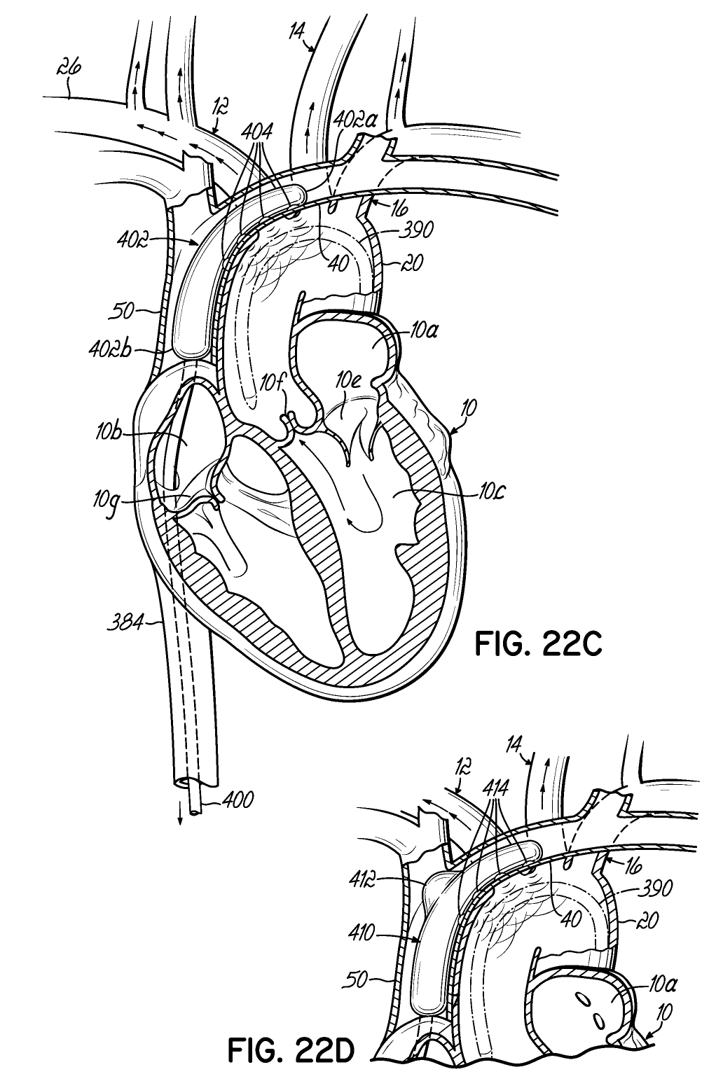

FIGS. 22A-22C illustrate a catheter delivered device for emitting ultrasonic energy in the vicinity of the aorta 20 to redirect or divert material in the blood stream away from the great vessels 12, 14, 16. As with the other embodiments of this invention, this embodiment may be implemented in many different ways. As one example, a catheter 400 may be directed through a femoral vein 382 into the inferior vena cava 384, the right side of the heart 10, and into the superior vena cava 50 and, as necessary, the innominate vein 40. The catheter 400 may carry and/or deploy an ultrasonic energy emitting device which carries an ultrasonic energy emission element 402 having one or more ultrasonic energy emitting transducers or elements 404. Element 402 may be expandable and such expansion may occur upon or after deployment from catheter 400. For example, a mechanically expandable element or balloon type element having a generally tapered or even a teardrop shape may be used. A generally tapered shape for element 402 is shown in FIG. 22C as one of many possible examples. The tapered element 402 may include ultrasonic emitting transducers 404 affixed thereto, as shown, for emitting ultrasonic energy at an appropriate location in the superior vena cava 50 and/or innominate vein 40 to redirect or divert material in the blood flowing through the aorta 20 away from at least one of the great vessels 12, 14, 16. For example, once the expandable element 402 is deployed as shown in FIG. 22C, the more tapered end 402a of the element 402 may be initially pushed into the innominate vein 40 until resistance is felt, and then pulled back a suitable distance so as to provide an opening for blood flow past the element 402. The element 402 may also or alternatively be precurved with a retained shape generally following the superior vena cava 50 and/or innominate vein 40 so that the element 402 is positively and accurately located and oriented, for example, as shown in FIG. 22C with the curve helping to ensure that the one or more ultrasonic transducers 404 are directed toward the aorta 20 with energy being emitted generally away from the origins of the great vessels 12, 14, 16.

For many patients, the most suitable location for the element 402 and the ultrasonic transducers 404 will be in the superior vena cava 50 generally at the junction with the innominate vein 40. It will be appreciated that the element 402 may include suitable flow passages, recesses, channels or the like to allow blood flow to occur past the element 402. As shown, an end 402b of the element having a larger diameter or width may reside in the superior vena cava 50 while the end 402a of the element 402 with the smaller diameter or width may reside in the innominate vein 40. The shape shown in FIG. 22C is merely representative and it will be appreciated that many other shapes may be used instead, including other tapered shapes, teardrop shapes and other shapes of various lengths, widths and tapers (if any). As shown in the views of FIGS. 22B and 22C, the element 402 may automatically expand when deployed from a suitable catheter 400 or, alternatively, the ultrasonic emission element 402 may be actively expanded by the surgeon when positioned at the desired location.

FIG. 22D illustrates an alternative embodiment to the element 402 shown in FIG. 22C. In this alternative embodiment, a similar element 410 may include a locating member 412 that provides resistance to further upward or inward pushing of the element 410. The surgeon then knows that the element 410 is properly positioned for directing ultrasonic energy with the emitters 414 through the aorta 20. Other types of locating members or structure may be used instead.

Again, with respect to FIGS. 22A-22D, another catheter 390 may be placed within the aorta 20 itself for various purposes, such as ultrasonic sensing, therapeutic procedures, additional ultrasonic diversion of material, or other reasons. As mentioned previously, the ultrasound emission could be combined with other catheters as a kit or system used for various procedures inside the aorta or the heart.

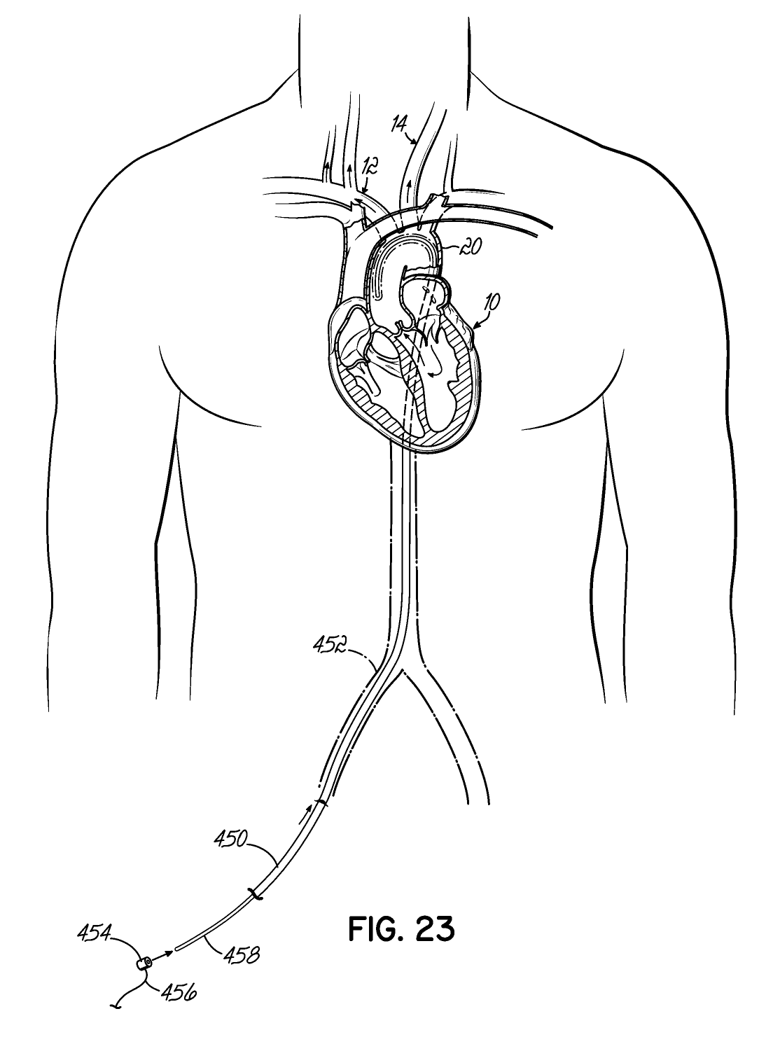

FIG. 23 illustrates a catheter 450 inserted via a femoral artery 452 and into the ascending aorta 20. An ultrasonic emitter or transducer 454 having a wire lead 456 is shown being threaded onto a guidewire 458. One or more of these ultrasonic emitters 454 may be inserted in this manner and located within the ascending aorta 20 in proximity to the origins of the great vessels 12, 14, 16, for example.

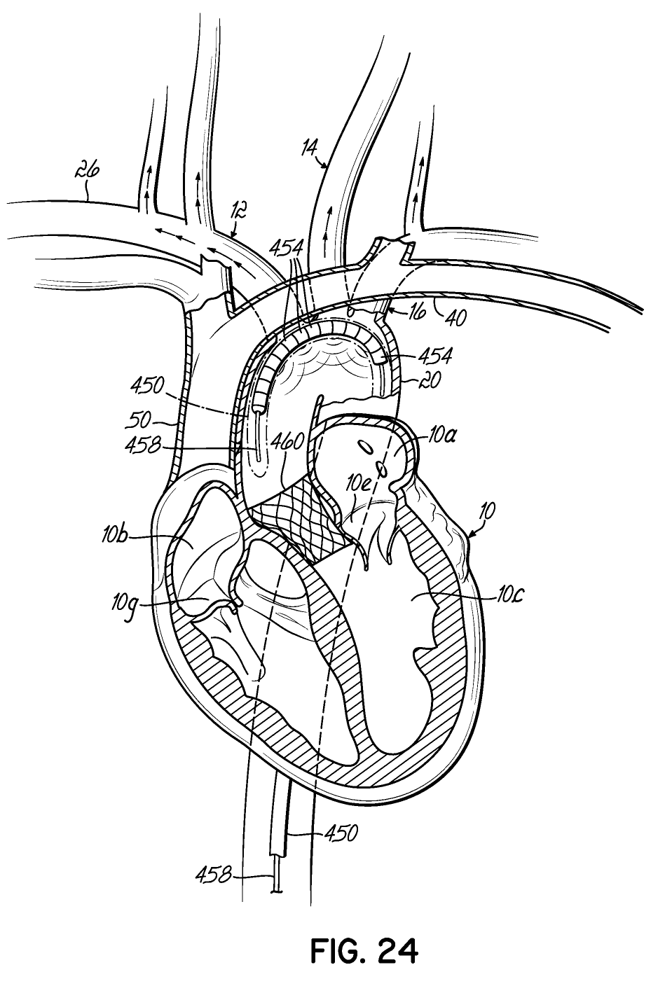

As a further example of the system and method shown in FIG. 23, FIG. 24 illustrates the placement of multiple ultrasonic emitters 454 to form a segmented array in the curved portion of the aorta 20 proximate to the origins of the great vessels 12, 14, 16. These ultrasonic emitters 454 may be threaded along the guidewire 458 as shown and contained within a sheath or catheter 450 shown in dash-dot lines in the curved portion of the aorta 20. The ultrasonic emitters 454 may be used in any of the manners described herein. FIG. 24 further illustrates a replacement aortic valve 460. As with any other embodiment contemplated herein, the ultrasonic emitters 454 may be used in conjunction with any other procedure and associated devices in the form of a kit or system for purposes of stroke prevention during at least the time when the patient is undergoing the procedure. Thus, the ultrasonic emitters 454 may be located within the curved portion of the aorta 20, for example, while an aortic valve replacement procedure is being performed in this particular example.

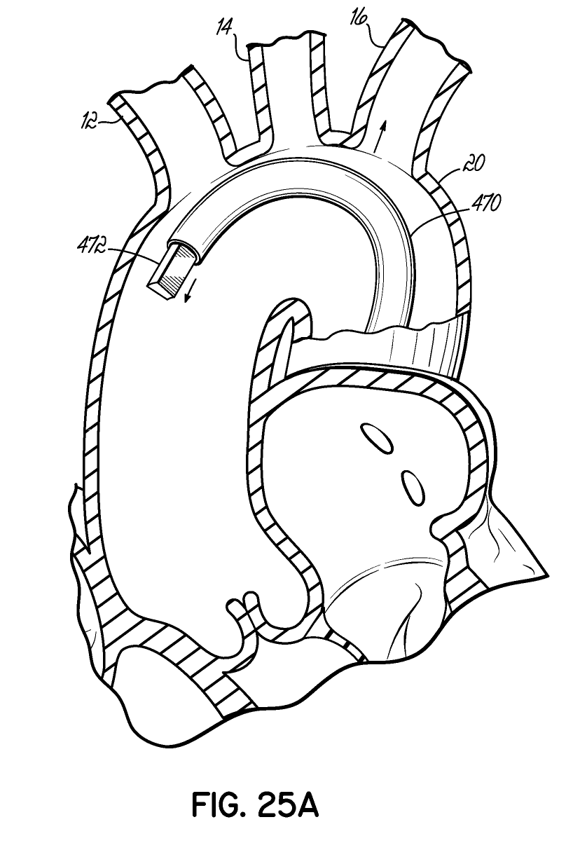

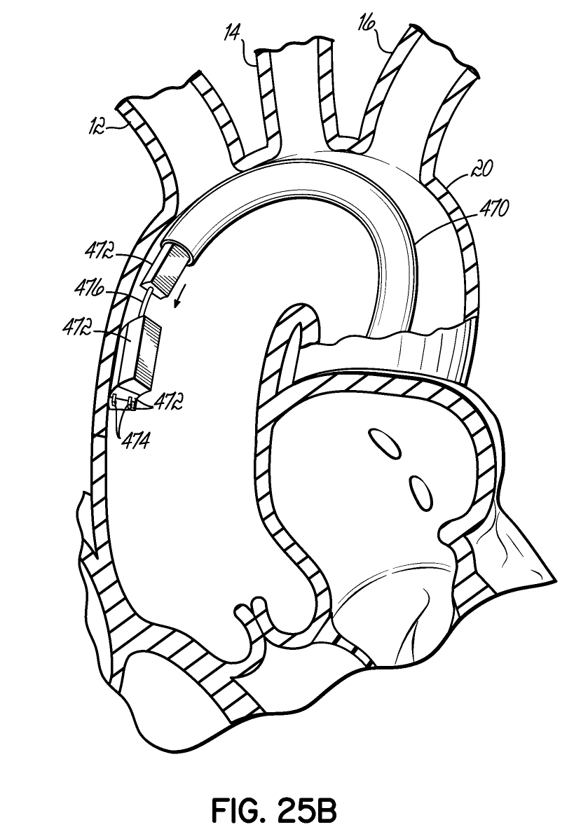

FIGS. 25A and 25B illustrate another embodiment of an ultrasonic emitting catheter device 470 which carries a plurality of ultrasonic emitters 472 contained within one or more lumens of the catheter or sheath 470 in a deployable fashion. As with other embodiments, the ultrasonic emitters 472 may be located in various vascular locations generally proximate to the origins of the great vessels 12, 14, 16, but are shown here in the curved portion of the aorta 20. The ultrasonic emitters 472 may be contained within the catheter or sheath 470 in a relatively low profile state and then deployed into a profile in which two or more of the emitters 472 are placed adjacent one another to form a larger surface area or profile, as shown in FIG. 25B. FIG. 25B illustrates three ultrasonic emitters 472 side-by-side and another ultrasonic emitter 472 being deployed through a distal end of the sheath or catheter 470. The larger profile or surface area formed by the assembled groups of ultrasonic emitters 472 will concentrate more ultrasonic energy at the deployed location while the lower profile state of the emitters 472 within the catheter or sheath 470 allow delivery to the desired location through smaller diameter sheaths or catheters.

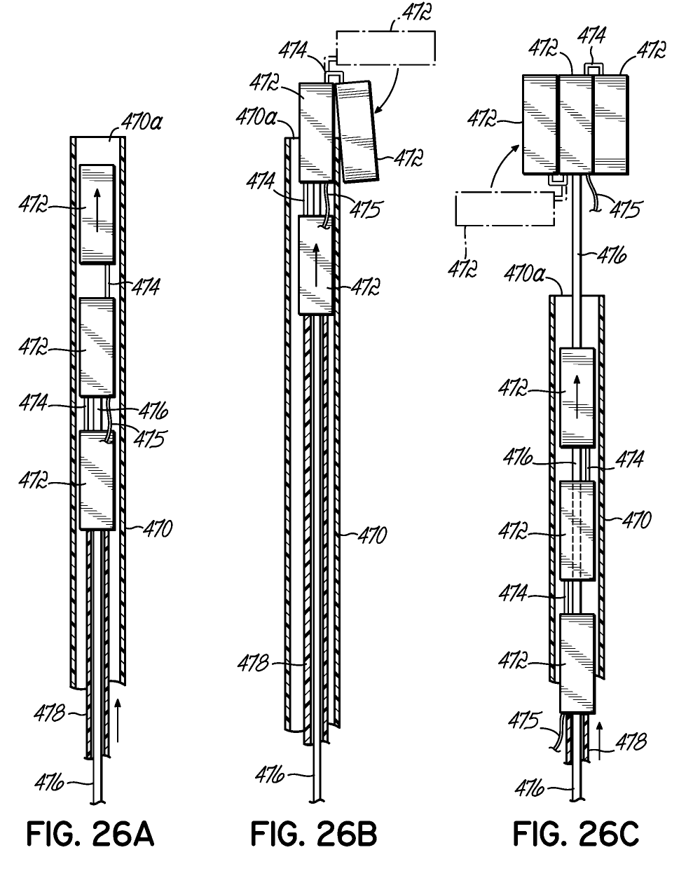

FIGS. 26A, 26B and 26C illustrate in further detail how the individual ultrasonic emitters 472 may be deployed through a catheter 470 and assembled together in a deployed and expanded state creating a higher profile or surface area. In this regard, Nitinol or other superelastic wire spring arms 474 incorporating or additionally including assembled power/control leads may comprise an expandable structure designed to couple the individual ultrasonic emitters 472 together. Of course, other expandable structures may be used instead. Once deployed outside the distal end 470a of the catheter 470, the Nitinol spring arms 474 may resume a shape that biases the ultrasonic emitters 472 into the position, for example, shown in FIG. 26C. The assembled or bundled ultrasonic emitters 472 may then be suitably fixed within the desired blood vessel, such as in one of the manners previously described. As mentioned, wire leads 475 may be used to supply power and control functions to the ultrasonic emitters. A guidewire 476 may be used to guide the ultrasonic emitters 472 within the catheter 470 and out from the distal end 470a. A pusher device 478, such as another smaller diameter catheter may be used to push the ultrasonic emitters 472 out from the distal end 470a of the catheter 470.

It will be appreciated that the various ultrasonic energy emission devices discussed herein may be placed and/or implanted in any manner and through any pathway in and through the patient and may be implanted for time periods of any duration. This includes percutaneous or so-called minimally invasive methods and any other methods of varying invasiveness, including open chest procedures. Of course, each embodiment may include any suitable power supply and controls and may be provided in any form of kit or system with or without other types of related medical devices, such as devices for performing diagnostic or therapeutic procedures in conjunction with ultrasonic diversion of material in the bloodstream.

While the present invention has been illustrated by a description of various preferred embodiments and while these embodiments have been described in some detail, it is not the intention of the Applicant to restrict or in any way limit the scope of the appended claims to such detail. Additional advantages and modifications will readily appear to those skilled in the art. The various features of the invention may be used alone or in any combination depending on the needs and preferences of the user. This has been a description of the present invention, along with the preferred methods of practicing the present invention as currently known. However, the invention itself should only be defined by the appended claims.

1. A system for directing ultrasonic energy through the blood flow in the aorta of a patient proximate the origins of the great vessels to the head of the patient to divert material in the blood flow from the origins of the great vessels, comprising:
a first device configured for introduction into the aorta of the patient and positioning proximate the origins of the great vessels to the head of the patient, said device including a catheter capable of being curved to follow the curvature of the aorta at the origins of the great vessels, and
at least one ultrasonic energy emitter carried by said first device and configured to deliver ultrasonic energy at therapeutic levels sufficient to divert material in the blood flowing through the aorta such that the material moves away from the origins of the great vessels.

2. The system of claim 1, further comprising a power supply and a control coupled together and further coupled with said ultrasonic energy emitter, said power supply providing power to operate said ultrasonic energy emitter and said control operative to regulate operation of said ultrasonic energy emitter.

3. The system of claim 2, wherein said control is capable of operating said ultrasonic energy emitter to deliver ultrasonic energy at a frequency in a range of about 20 kHz to about 45 kHz.

4. The system of claim 3, wherein said control is operative to allow cycling of power to said emitters.

5. The system of claim 1, further comprising:
a second device configured to perform a procedure within the cardiovascular system and associated for use with said first device.

6. The system of claim 5, wherein said second device further comprises a cardiovascular diagnostic device or a cardiovascular therapeutic device.

7. The system of claim 1, further comprising a second device configured to perform a therapeutic or diagnostic procedure in the cardiovascular system, wherein one of said first or second devices further carries or guides the other of said first or second devices.

8. A system for directing ultrasonic energy through the blood flow in the aorta of a patient proximate the origins of the great vessels to the head of the patient to divert material in the blood flow from the origins of the great vessels, comprising:
a first device configured for introduction into the cardiovascular system of the patient and positioning proximate the origins of the great vessels to the head of the patient, and
at least one ultrasonic energy emitter carried by said first device and configured to deliver ultrasonic energy at therapeutic levels sufficient to divert material in the blood flowing through the aorta such that the material moves away from the origins of the great vessels.

9. The system of claim 8, further comprising a power supply and a control coupled together and further coupled with said ultrasonic energy emitter, said power supply providing power to operate said ultrasonic energy emitter and said control operative to regulate operation of said ultrasonic energy emitter.

10. The system of claim 9, wherein said control is capable of operating said ultrasonic energy emitter to deliver ultrasonic energy at a frequency in a range of about 20 kHz to about 45 kHz.

11. The system of claim 8, wherein said control is operative to allow cycling of power to said emitters.

12. The system of claim 8, further comprising:
a second device configured to perform a procedure within the cardiovascular system and associated for use with said first device.

13. The system of claim 8, wherein said second device further comprises a cardiovascular diagnostic device or a cardiovascular therapeutic device.

14. The system of claim 8, further comprising a second device configured to perform a therapeutic or diagnostic procedure in the cardiovascular system, wherein one of said first or second devices further carries or guides the other of said first or second devices.

15. A method of directing ultrasonic energy in the region proximate the origins of the great vessels to the head of the patient to divert material in the blood flow away from the origins of the great vessels, comprising:
directing a device carrying at least one ultrasonic energy emitter into the cardiovascular system of the patient,
locating the ultrasonic energy emitter in the region proximate the origins of the great vessels to the head of the patient, and
activating the ultrasonic energy emitter to direct ultrasonic energy in a direction effective to divert the material away from the origins of the great vessels.

16. The method of claim 15, wherein locating the ultrasonic energy emitter further comprises positioning the ultrasonic energy emitter in at least one of: the innominate vein, superior vena cava, or the right side of the heart.

17. The method of claim 15, further comprising:
performing at least one of a diagnostic procedure or a therapeutic procedure in the cardiovascular system while the ultrasonic energy emitter is activated.

18. The method of claim 17, wherein the diagnostic or therapeutic procedure further comprises at least one of: a repair procedure on a heart valve, a heart catheterization procedure, an aortic catheterization procedure, a blood procedure, a blood pump installation procedure, a procedure for correcting a septal defect in the heart, a heart valve replacement procedure, an arrhythmia diagnostic procedure or an arrhythmia treatment procedure.

19. A method of directing ultrasonic energy in the region proximate the origins of the great vessels to the head of the patient to divert material in the blood flow away from the origins of the great vessels, comprising:
directing a device carrying at least one ultrasonic energy emitter into the aorta of the patient,
locating the ultrasonic energy emitter in the region of the aorta proximate the origins of the great vessels to the head of the patient, and
activating the ultrasonic energy emitter to direct ultrasonic energy from within the aorta and in a direction effective to divert the material in the blood flow away from the origins of the great vessels.

20. A device for directing ultrasonic energy through blood flowing in the aorta of a patient proximate the origins of the great vessels to the head of the patient to divert material in the blood flow from the origins of the great vessels, comprising:
an expandable structure configured to be delivered into a blood vessel and having one or more expandable portions to position said expandable structure relative to the blood vessel, and
an ultrasonic energy emitter carried by said expandable structure.

21. The device of claim 20, wherein said expandable structure comprises a stent-like device.

22. The device of claim 20, wherein said expandable structure comprises a balloon.

23. The device of claim 20, wherein said expandable structure comprises a structure coupling a plurality of assembled ultrasonic energy emitters together.

24. A device for directing ultrasonic energy through blood flowing in the aorta of a patient proximate the origins of the great vessels to the head of the patient to divert material in the blood flow from the origins of the great vessels, comprising:
an element configured to be delivered into a blood vessel, said element including a curve that is retained for purposes of positioning and orienting said element in a curved section of the blood vessel, and
an ultrasonic energy emitter carried by said element.

25. An implantable fuel cell for use with an ultrasonic energy emitting device implantable within the cardiovascular system of a patient.

26. A vascular graft adapted to provide protection against an embolus traveling to the brain of a patient, the graft comprising:
vascular graft material, and
at least one ultrasonic energy emitter carried by the vascular graft material.

27. The vascular graft of claim 26, wherein the vascular graft material is formed as an aortic graft.

28. A stent-like device adapted to provide protection from an embolus traveling to the brain of a patient, comprising:
an expandable stent-like structure capable of moving from a contracted position for delivery into the vascular system of the patient to an expanded position for implantation in the vascular system, and
at least one ultrasonic energy emitter carried by the expandable stent-like structure.

29. A implant for cooling blood in a patient, comprising:
a device configured to be implanted in the cardiovascular system of the patient with at least a portion of the device in contact with blood flowing within the cardiovascular system; and
a cooling system operatively coupled with the device and operable to remove heat from the blood contacting at least the portion of the device.
